(19) United States
(12) Patent Application Publication
(10) Pub. No.: US 2023/0365479 A1
(43) Pub. Date: Nov. 16, 2023

(54) Processes for Converting C8 Aromatic Hydrocarbons

(71) Applicant: EXXONMOBIL CHEMICAL PATENTS INC., Baytown, TX (US)

(72) Inventors: Eric D. Metzger, Alameda, CA (US); Ali A. Kheir, Houston, TX (US); Maria Milina, Glen Ridge, NJ (US); Kathleen M. Keville, Beaumont, TX (US)

(21) Appl. No.: 18/044,584

(22) PCT Filed: Sep. 1, 2021
(86) PCT No.: PCT/US2021/048633

§ 371 Date: Mar. 9, 2023

Related U.S. Application Data
(60) Provisional application No. 63/085,288, filed on Sep. 30, 2020.

(51) Int. Cl. C07C 5/27; B01J 29/40; B01J 35/10

(52) U.S. Cl. CPC C07C 5/2708; B01J 29/40; B01J 35/1019; B01J 35/1023; B01J 2229/42; C07C 2529/40

(57) ABSTRACT

Processes for converting C8 aromatic hydrocarbons. In some embodiments, a process for converting a hydrocarbon feed that can include C8 aromatic hydrocarbons can include feeding the hydrocarbon feed into a conversion zone and contacting the hydrocarbon feed at least partly in a liquid phase with an isomerization catalyst composition in the conversion zone under conversion conditions to effect isomerization of at least a portion of the C8 aromatic hydrocarbons to produce a conversion product rich in para-xylene. In some embodiments, the isomerization catalyst composition can include a zeolite (preferably a ZSM-5 zeolite) that can have a silica (SiO₂) to alumina (AI₂O₃) molar ratio of 10 to 100, a total surface area of 200 m²/g to 700 m²/g, a micropore surface area of 50 m²/g to 600 m²/g, and an external surface area of 55 m²/g to 550 m²/g.

### CROSS-REFERENCE TO RELATED APPLICATION

This application claims priority to and the benefit of U.S. Provisional Application No. 63/085,288 having a filing date of Sep. 30, 2020, the disclosure of which is incorporated herein by reference in its entirety.

### FIELD

This disclosure relates to processes for converting C8 aromatic hydrocarbons. More particularly, this disclosure relates to processes for isomerization of meta-xylene and/or ortho-xylene to produce para-xylene. This disclosure is useful, e.g., in making para-xylene products from a mixed C8 aromatic hydrocarbon feed, particularly a mixed xylenes feed comprising para-xylene at a below equilibrium concentration.

### BACKGROUND

A high purity para-xylene product is typically produced by separating para-xylene from a para-xylene-rich aromatic hydrocarbon mixture that includes para-xylene, ortho-xylene, meta-xylene, and sometimes ethylbenzene in a para-xylene separation/recovery system. The para-xylene recovery system can include, e.g., a crystallizer and/or an adsorption chromatography separation system known in the prior art. A para-xylene-depleted effluent recovered from the para-xylene recovery system (the “filtrate” from a crystallizer upon separation of the para-xylene crystals, or the “raffinate” from the adsorption chromatography separating system, collectively “raffinate” in this disclosure) is rich in meta-xylene and ortho-xylene, and contains para-xylene at a concentration typically below its concentration in an equilibrium mixture consisting of meta-xylene, ortho-xylene, and para-xylene. To increase the yield of para-xylene, the raffinate stream can be fed into an isomerization unit, where the xylenes undergo isomerization reactions by contacting an isomerization catalyst to produce an isomerized effluent rich in para-xylene as compared to the raffinate. At least a portion of the isomerized effluent, after optional separation and removal of lighter hydrocarbons that can be produced in the isomerization unit, can be recycled to the para-xylene recovery system, forming a “xylenes loop”.

Conventionally xylenes isomerization has been carried out under conditions where the C8 aromatic hydrocarbons are substantially in the vapor phase in the presence of the isomerization catalyst (vapor-phase isomerization, or “VPI”). Newer technology, however, has been developed to allow xylenes isomerization at a significantly lower temperature in the presence of the isomerization catalyst, where the C8 aromatic hydrocarbons are at least partly and preferably substantially in the liquid phase (liquid-phase isomerization, or “LPI”). The use of liquid phase isomerization vs. vapor phase isomerization can reduce the number of phase changes (liquid to/from vapor) required to process the C8 aromatic feed. This provides the process with sustainability advantages in the form of significant energy savings. It would be highly advantageous for any para-xylene production plant to deploy a liquid phase isomerization unit, in addition to or in lieu of a vapor phase isomerization unit.

Due to the advantages of the liquid phase isomerization process, improvements in this technology, particularly the isomerization catalyst used, are also needed. This disclosure satisfies this and other needs.

### SUMMARY

It has been found that by treating a precursor catalyst composition such as one comprising a zeolite to increase its external surface area, an isomerization catalyst composition can be made to have a substantially higher para-xylene selectivity in a C8 aromatic hydrocarbon isomerization process.

Thus, a first aspect of this disclosure relates to a process for converting a hydrocarbon feed that can include C8 aromatic hydrocarbons. In some embodiments, the process can include feeding the hydrocarbon feed into a conversion zone and contacting the hydrocarbon feed at least partly in a liquid phase with an isomerization catalyst composition in the conversion zone under conversion conditions to effect isomerization of at least a portion of the C8 aromatic hydrocarbons to produce a conversion product rich in para-xylene. In some embodiments, the isomerization catalyst composition can include a zeolite (e.g., preferably a ZSM-5 zeolite) that can have a silica (SiO₂) to alumina (Al₂O₃) molar ratio of 10 to 100, a total surface area of 200 m²/g to 700 m²/g, a micropore surface area of 50 m²/g to 600 m²/g, and an external surface area of 55 m²/g to 550 m²/g.

A second aspect of this disclosure relates to a process for converting an aromatic hydrocarbon. In some embodiments, the process can include feeding a hydrocarbon feed that can include C8 aromatic hydrocarbons into a conversion zone and contacting the hydrocarbon feed with a catalyst that can include a ZSM-5 zeolite in the conversion zone under conversion conditions to effect isomerization of at least a portion of the C8 aromatic hydrocarbons to produce a conversion product rich in para-xylene. The conversion conditions can include an absolute pressure sufficient to maintain the C8 aromatic hydrocarbons in a liquid phase, a weight hour space velocity of 1 hr⁻¹ to 15 hr⁻¹, and a temperature of 150° C. to 300° C. The isomerization catalyst composition can include a ZSM-5 zeolite having a silica (SiO₂) to alumina (Al₂O₃) molar ratio of 20 to 40, a total surface area of 400 m²/g to 500 m²/g, a micropore surface area of 300 m²/g to 450 m²/g, and an external surface area of 100 m²/g to 200 m²/g.

A third aspect of this disclosure relates to a process for converting a hydrocarbon feed that can include C8 aromatic hydrocarbons. In some embodiments, the process can include providing a precursor catalyst composition that can exhibit a first external surface area of a1m²/g and treating the precursor catalyst composition to obtain a treated precursor catalyst composition. The treated precursor catalyst composition can exhibit a second external surface area of a2 m²/g. In some embodiments, (a2−a1)/a1*100% can be ≥10%. The process can also include forming an isomerization catalyst composition from the treated precursor catalyst composition. The process can also include feeding the hydrocarbon feed into a conversion zone and contacting the hydrocarbon feed at least partly in a liquid phase with the isomerization catalyst composition in the conversion zone under conversion conditions to effect isomerization of at least a portion of the C8 aromatic hydrocarbons to produce a conversion product rich in para-xylene.

### DETAILED DESCRIPTION

Various specific embodiments, versions and examples of the invention will now be described, including preferred embodiments and definitions that are adopted herein for purposes of understanding the claimed invention. While the following detailed description gives specific preferred embodiments, those skilled in the art will appreciate that these embodiments are exemplary only, and that the invention may be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

In this disclosure, a process is described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other steps, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for acquiring the measurement.

Certain embodiments and features are described herein using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated.

The indefinite article “a” or “an”, as used herein, means “at least one” unless specified to the contrary or the context clearly indicates otherwise. Thus, embodiments using “a reactor” or “a conversion zone” include embodiments where one, two or more reactors or conversion zones are used, unless specified to the contrary or the context clearly indicates that only one reactor or conversion zone is used.

The term “hydrocarbon” means (i) any compound consisting of hydrogen and carbon atoms or (ii) any mixture of two or more such compounds in (i). The term “Cn hydrocarbon,” where n is a positive integer, means (i) any hydrocarbon compound comprising carbon atom(s) in its molecule at the total number of n, or (ii) any mixture of two or more such hydrocarbon compounds in (i). Thus, a C2 hydrocarbon can be ethane, ethylene, acetylene, or mixtures of at least two of these compounds at any proportion. A “Cm to Cn hydrocarbon” or “Cm-Cn hydrocarbon,” where m and n are positive integers and m<n, means any of Cm, Cm+1, Cm+2, . . . , Cn−1, Cn hydrocarbons, or any mixtures of two or more thereof. Thus, a “C2 to C3 hydrocarbon” or “C2-C3 hydrocarbon” can be any of ethane, ethylene, acetylene, propane, propene, propyne, propadiene, cyclopropane, and any mixtures of two or more thereof at any proportion between and among the components. A “saturated C2-C3 hydrocarbon” can be ethane, propane, cyclopropane, or any mixture thereof of two or more thereof at any proportion. A “Cn+ hydrocarbon” means (i) any hydrocarbon compound comprising carbon atom(s) in its molecule at the total number of at least n, or (ii) any mixture of two or more such hydrocarbon compounds in (i). A “Cn− hydrocarbon” means (i) any hydrocarbon compound comprising carbon atoms in its molecule at the total number of at most n, or (ii) any mixture of two or more such hydrocarbon compounds in (i). A “Cm hydrocarbon stream” means a hydrocarbon stream consisting essentially of Cm hydrocarbon(s). A “Cm-Cn hydrocarbon stream” means a hydrocarbon stream consisting essentially of Cm-Cn hydrocarbon(s).

“Crystallite” means crystalline grain of a material. Crystallites with microscopic or nanoscopic size can be observed using microscopes such as transmission electron microscope (“TEM”), scanning electron microscope (“SEM”), reflection electron microscope (“REM”), scanning transmission electron microscope (“STEM”), and the like. Crystallites can aggregate to form a polycrystalline material.

For the purposes of this disclosure, the nomenclature of elements is pursuant to the version of Periodic Table of Elements as described in CHEMICAL AND ENGINEERING NEWS, 63(5), pg. 27 (1985).

The term “aromatic” is to be understood in accordance with its art-recognized scope, which includes alkyl substituted and unsubstituted mono- and polynuclear compounds.

The term “rich” when used in phrases such as “X-rich” or “rich in X” means, with respect to an outgoing stream obtained from a device, e.g., a conversion zone, that the stream comprises material X at a concentration higher than in the feed material fed to the same device from which the stream is derived. The term “lean” when used in phrases such as “X-lean” or “lean in X” means, with respect to an outgoing stream obtained from a device, e.g., a conversion zone, that the stream comprises material X at a concentration lower than in the feed material fed to the same device from which the stream is derived.

The terms “para-xylene selectivity” and “pX selectivity” are used interchangeably and refer to the para-xylene concentration among all xylenes in a conversion product or conversion product rich in para-xylene.

The term “comparable para-xylene selectivity” means the para-xylene selectivity for each of two given examples is within about 2 percent of one another. For example, a first product that has a para-xylene selectivity of 20% would have a comparable para-xylene selectivity relative to a second product that has a para-xylene selectivity of +/−0.4%, i.e., 19.6% to 20.4%.

The term “comparable ethylbenzene conversion” means the conversion of ethylbenzene for each of two given examples is within 1 percentage point or less of one another. For example, a first product that has an ethylbenzene conversion of 4% would have a comparable ethylbenzene conversion relative to a second product that has an ethylbenzene conversion of +/−1%, i.e., 3% to 5%.

The term “xylenes loss” (“Lx(1)”) can be calculated as Lx(1)=100%*(W1−W2)/W1, where W1 is the aggregate weight of all xylenes present in the hydrocarbon feed that includes C8 aromatics, and W2 is the aggregate weight of all xylenes present in the conversion product.

The terms “liquid phase isomerization” and “LPI” interchangeably mean isomerization under isomerization conditions such that ≥20 wt %, preferably ≥30 wt %, preferably ≥40 wt %, preferably ≥50 wt %, preferably ≥60 wt %, preferably ≥70 wt %, preferably ≥80 wt %, preferably ≥90 wt %, or preferably ≥95 wt %, of the C8 aromatic hydrocarbons in the isomerization zone is present in liquid phase. In certain embodiments of LPI, >98 wt % (substantially all) of C8 aromatic hydrocarbons are present in liquid phase in the isomerization zone.

The terms “micropore”, “mesopore”, and “macropore” refer to pores having an average cross-sectional length (diameter if circular) of less than 2 nm, 2 nm to 50 nm, and greater than 50 nm, respectively.

The term “micropore surface area” refers to the surface area of a given sample attributable to pores having an average cross-sectional length (diameter if circular) of less than 2 nm. The term “mesopore surface area” refers to the surface area of a given sample attributable to pores having an average cross-sectional length (diameter if circular) of 2 nm to 50 nm. The term “macropore surface area” refers to the surface area of a given sample attributable to pores having an average cross-sectional length (diameter if circular) of greater than 50 nm.

The term “external surface area” is the total surface area of a given sample minus the micropore surface area of that sample and, as such, is equal to the sum of the mesopore surface area and the macropore surface area. The total surface area and the micropore surface area can be measured via the well-known Brunauer-Emmett-Teller (BET) method. The total surface area and the t-Plot micropore surface area can be measured by nitrogen adsorption/desorption after degassing of the extrudate for 4 hours at 350° C. As noted above, the external surface area can be obtained by subtracting the t-plot micropore surface area from the total surface area. More information regarding the method can be found, for example, in “Characterization of Porous Solids and Powders: Surface Area, Pore Size and Density”, S. Lowell et al., Springer, 2004.

In this disclosure, NH₄F·HF means a mixture of NH₄F and HF at any suitable proportion between them. One preferred example of NH₄F·HF is a mixture of NH₄F and HF at a 1:1 molar ratio between them.

### I. The First Aspect of This Disclosure

### I.1 Overview

In some embodiments, a hydrocarbon feed that includes C8 aromatic hydrocarbons, e.g., meta-xylene and/or ortho-xylene, can be contacted while at least partly in a liquid phase with a catalyst that includes a zeolite within a conversion zone under conversion conditions to effect isomerization of at least a portion of any meta-xylene, at least a portion of any ortho-xylene, or both to produce a conversion product rich in para-xylene. In some embodiments, the zeolite can have a total surface area of 200 m²/g to 700 m²/g, a micropore surface area of 50 m²/g to 600 m²/g, and an external surface area of 55 m²/g to 550 m²/g. In other embodiments, the zeolite can have a total surface area of 300 m²/g to 500 m²/g, a micropore surface area of ≥300 m²/g, and an external surface area of 100 m²/g to 200 m²/g.

Non-limiting examples of useful zeolites in the processes of this disclosure include: ZSM-5, ZSM-11, ZSM-5 and ZSM-11 intergrowth, ZSM-22, ZSM-23, ZSM-35, ZSM-48, a MWW framework zeolite such as MCM-22, MCM-36, MCM-49, MCM-56, PSH-3, SSZ-25, ERB-1, ITQ-1, ITQ-2, UZM-8, UZM-8HS, and mixtures and combinations thereof. A preferred zeolite is a ZSM-5 zeolite.

It has been surprisingly and unexpectedly discovered that by replacing a conventional isomerization catalyst that includes a zeolite (e.g., a ZSM-5 zeolite) that has an external surface area of <60 m²/g, with the isomerization catalyst composition that includes the modified ZSM-5 zeolite that can have an external surface area of ≥120 m²/g, a significant increase in the para-xylene selectivity in the conversion product can be obtained while significantly increasing the weight hour space velocity (WHSV), e.g., double the WHSV, as compared to the conventional catalyst when operated at a comparable ethylbenzene conversion.

### I.2 ZSM-5 Zeolite

The isomerization catalyst composition that includes the ZSM-5 zeolite can include 1 wt %, 5 wt %, 10 wt %, 20 wt %, 30 wt %, or 40 wt % to 60 wt %, 70 wt %, 80 wt %, 90 wt %, or 100 wt % of the ZSM-5 zeolite, based on a total weight of the isomerization catalyst composition.

The ZSM-5 zeolite can have a silica (SiO₂) to alumina (Al₂O₃) molar ratio of 10, 15, 20, 25, 30, 35, or 40 to 50, 75, 100, 125, 150, 175, or 200. In some embodiments, the ZSM-5 zeolite can have a silica to alumina molar ratio of 15 to 200, 15 to 150, 15 to 100, 15 to 75, 15 to 50, 20 to 200, 20 to 150, 20 to 100, 20 to 75, 20 to 50, 30:200, 30 to 150, 30 to 100, 30 to 75, or 30 to 50. The silica to alumina molar ratio refers to the molar ratio in the rigid anionic framework of the zeolite and excludes any silicon (silicon metal and/or silica) and aluminum (aluminum metal and/or alumina) in a binder, e.g., when the zeolite is included as a component of an extrudate, or in cationic or other form within the channels of the zeolite. The silica to alumina molar ratio can be determined by conventional analysis, e.g., inductively coupled plasma mass spectrometry (ICP-MS) or X-ray fluorescence (XRF).

In some embodiments, the ZSM-5 zeolite can have an alpha value of 1 to 5,000, 500 to 3,000, 750 to 2,750, or 1,000 to 2,500 and a silica to alumina molar ratio of 15 to 200, 15 to 150, 15 to 100, 15 to 75, 15 to 50, 20 to 200, 20 to 150, 20 to 100, 20 to 75, 20 to 50, 30:200, 30 to 150, 30 to 100, 30 to 75, or 30 to 50. In other embodiments, the ZSM-5 zeolite can have an alpha value of 1 to 5,000, 500 to 3,000, 750 to 2,750, or 1,000 to 2,500 and a silica to alumina molar ratio of 15, 20, 25, 30, or 35 to 40, 50, 70, 100, 150, or 200.

The ZSM-5 zeolite can have a total surface area of 100 m²/g, 150 m²/g, 200 m²/g, 250 m²/g, or 300 m²/g to 400 m²/g, 500 m²/g, 600 m²/g, 700 m²/g, 800 m²/g, 900 m²/g, or 1,000 m²/g. In some embodiments, the ZSM-5 zeolite can have a total surface area of 150 m²/g to 1,000 m²/g, 200 m²/g to 600 m²/g, or 300 m²/g to 500 m²/g.

The ZSM-5 zeolite can have a micropore surface area of 50 m²/g, 75 m²/g, 100 m²/g, or 150 m²/g to 200 m²/g, 300 m²/g, 400 m²/g, 500 m²/g, or 600 m²/g. In some embodiments, the ZSM-5 zeolite can have a micropore surface area of ≥50 m²/g to 600 m²/g, ≥100 m²/g to 600 m²/g, ≥150 m²/g to 600 m²/g, ≥50 m²/g to 400 m²/g, ≥100 m²/g to 400 m²/g, ≥150 m²/g to 400 m²/g.

The ZSM-5 zeolite can have an external surface area of 1 m²/g, 10 m²/g, 20 m²/g, 30 m²/g, 40 m²/g, 50 m²/g, 75 m²/g, 100 m²/g, 125 m²/g, or 150 m²/g to 300 m²/g, 400 m²/g, 500 m²/g, 600 m²/g, 700 m²/g, 800 m²/g, 900 m²/g, or 950 m²/g. In some embodiments, the ZSM-5 zeolite can have an external surface area of 10 m²/g to 950 m²/g, 50 m²/g to 500 m²/g, 100 m²/g to 450 m²/g, 100 m²/g to 300 m²/g, 120 m²/g to 950 m²/g or 120 m²/g to 350 m²/g.

In some embodiments, the ZSM-5 zeolite can have a total surface area of 100 m²/g to 1,000 m²/g, a micropore surface area of 50 m²/g to 600 m²/g, and an external surface area of 1 m²/g to 950 m²/g. In other embodiments, the ZSM-5 zeolite can have a total surface area of 150 m²/g to 1,000 m²/g, a micropore surface area of 50 m²/g to 900 m²/g, and an external surface area of 100 m²/g to 950 m²/g. In other embodiments, the ZSM-5 zeolite can have a total surface area of 200 m²/g to 600 m²/g, a micropore surface area of 100 m²/g to 900 m²/g, and an external surface area of 100 m²/g to 900 m²/g. In still other embodiments, the ZSM-5 zeolite can have a total surface area of 150 m²/g to 800 m²/g, a micropore surface area of 100 m²/g to 700 m²/g, and an external surface area of 100 m²/g to 700 m²/g. In other embodiments, the ZSM-5 zeolite can have a total surface area of 200 m²/g to 600 m²/g, a micropore surface area of 100 m²/g to 500 m²/g, and an external surface area of 100 m²/g to 500 m²/g. In other embodiments, the ZSM-5 zeolite can have a total surface area of 200 m²/g to 600 m²/g, a micropore surface area of 50 m²/g or100 m²/g to 500 m²/g, and an external surface area of 120 m²/g to 500 m²/g.

### I3. Process for Making the ZSM-5 Zeolite

Parent ZSM-5 zeolite can be made via any suitable process or obtained from a suitable vendor. In some embodiments, the inventive mesoporous ZSM-5 zeolites were prepared by alkaline treatments of a parent zeolite that were carried out in 0.3 M aqueous NaOH (1 g of zeolite per 30 cm³ of solution). In a typical experiment, the alkaline solution was heated to 65° C., after which the parent zeolite sample was introduced. The resulting suspension was left to react for 30 min, followed by quenching, filtration, extensive washing using distilled water, and overnight drying at 65° C. Some samples were subsequently acid treated in 0.3 M aqueous HCl (1 g zeolite per 100 cm³ of solution) at 65° C. for 6 h. Prior to catalytic testing, the zeolites were converted into the protonic form by three consecutive ion exchanges in 0.1 M aqueous NH₄NO₃ (25° C., 12 h, 1 g zeolite per 100 cm³ of solution), followed by calcination at 550° C. for 5 h in static air using a ramp rate of 5° C./min.

### I.4 The Isomerization Catalyst Composition

In some embodiments, the ZSM-5 zeolite can be used directly as a catalyst, i.e., the ZSM-5 zeolite can be substantially free of any other component other than the ZSM-5 zeolite.

In such embodiment, the ZSM-5 zeolite can be a self-supported catalyst composition. In some embodiments, the ZSM-5 zeolite can be combined with a second zeolite, such as zeolites having a 10- or 12-member ring structure in their crystallites. Non-limiting examples of second zeolites can be or can include, but are not limited to, ZSM-11, ZSM-12, ZSM-22, ZSM-23, ZSM-35, ZSM-48, ZSM-57, ZSM-58, or any mixture thereof. In some embodiments, the second zeolite, if present, can be or can include one or more of the zeolites described in U.S. Pat. No. 3,702,886; RE29,948; U.S. Pat. Nos. 3,832,449; 4,556,477; 4,076,842; 4,016,245); 4,397,827); and 4,417,780.

If one or more second zeolites are included in the isomerization catalyst composition, the isomerization catalyst composition can include 1 wt %, 5 wt %, 10 wt %, 20 wt %, 30 wt %, or 40 wt % to 60 wt %, 70 wt %, 80 wt %, 90 wt %, or 99 wt % of the ZSM-5 zeolite, based on the total weight of the ZSM-5 zeolite and the one or more second zeolites. When the isomerization catalyst composition includes multiple second zeolites, each second zeolite can be present in any amount with respect to one another.

In some embodiments, the ZSM-5 zeolite can be combined with the second zeolite, e.g., the ZSM-11 zeolite, via simple mixing. In other embodiments, the ZSM-5 zeolite and the second zeolite, e.g., the ZSM-11 zeolite, can be a ZSM-5/second zeolite intergrowth zeolite, e.g., a ZSM-5/ZSM-11 intergrowth zeolite. In some embodiments, the ZSM-5/second zeolite intergrowth zeolite can include 1 wt %, 10 wt %, 20 wt %, or 40 wt % to 50 wt %, 70 wt %, 90 wt %, or 99 wt % of the ZSM-5 zeolite, based on a combined weight of the ZSM-5 zeolite and the second zeolite. Some ZSM-5/ZSM-11 intergrowth zeolites are disclosed in G. A. Jablonski, L. B. Sand, and J. A. Gard, Zeolites, Vol. 6, Issue 5, pgs. 396-402 (1986) and G. R. Millward, S. Ramdas, J. M. Thomas, and M. T. Barlow, J. Chem. Soc., Faraday Trans. 2, 1983, 79, 1075-1082.

In some embodiments, the ZSM-5 zeolite can be compounded with one or more other components or materials, e.g., binders, which serve as a support and/or provide additional hardness to the finished catalyst. The binder can serve as a diluent to control the amount of conversion in a given process so that products can be obtained in an economic and orderly manner without employing other means for controlling the rate of reaction.

Binders can be or can include, but are not limited to, alumina, silica, titania, zirconia, zirconium silicate, kaolin, one or more chromium oxides, other refractory oxides and refractory mixed oxides, and mixtures and combinations thereof. In some embodiments, the ZSM-5 zeolite can be composited with a porous binary matrix material such as silica-alumina, silica-magnesia, silica-zirconia, silica-thoria, silica-beryllia, silica-titania as well as ternary matrix material such as silica-alumina-thoria, silica-alumina-zirconia silica-alumina-magnesia and silica-magnesia-zirconia. Other suitable binder materials can be or can include, but are not limited to, naturally occurring clays, e.g., montmorillonite, bentonite, subbentonite and kaolin such as the kaolins commonly known as Dixie, McNamee, Georgia, and Florida clays or others in which the main mineral constituent is halloysite, kaolinite, nacrite, or anauxite, to improve the crush strength of the isomerization catalyst composition under commercial operating conditions. Such clays can be used in the raw state as originally mined or after being subjected to calcination, acid treatment, and/or chemical modification.

In some embodiments, the ZSM-5 zeolite can be combined with a hydrogenating component, such as tungsten, vanadium, molybdenum, rhenium, nickel, cobalt, chromium, manganese, or a noble metal such as platinum or palladium where a hydrogenation-dehydrogenation function is to be performed. Such component can be incorporated in the composition by way of co-crystallization, exchanged into the composition to the extent a Group IIIA element, e.g., aluminum, is in the structure, impregnated therein or physically admixed therewith. Such component can be impregnated in or onto the ZSM-5 zeolite such as, for example, in the case of platinum, treating the ZSM-5 zeolite with a solution containing a platinum metal-containing ion. Thus, suitable platinum compounds for this purpose include chloroplatinic acid, platinous chloride and various compounds containing the platinum amine complex. Combinations of metals and methods for their introduction can also be used.

In some embodiments, the ZSM-5 zeolite can be used in the form of an extrudate with a binder. The extrudate can be formed by extruding a mixture of the isomerization catalyst composition that is or includes the ZSM-5 zeolite and the binder. In some embodiments, the extrudate can be dried and calcined. It should be understood that the isomerization catalyst composition that includes the ZSM-5 zeolite can take any shape: cylinder, solid sphere, trilobe, quadrulobe, eggshell sphere, and the like. In some embodiments, the isomerization catalyst composition that includes the ZSM-5 zeolite, e.g., the ZSM-5 zeolite alone, an extrudate that includes the ZSM-5 zeolite, and/or the ZSM-5 zeolite and one or more second zeolites, can be ground into a powder and used as such.

In some embodiments, the binder in the isomerization catalyst composition that includes the ZSM-5 zeolite can be relatively high surface area binders, such as alumina and/or silica having a specific area of ≥200 m²/g or ≥250 m²/g. In other embodiments, the binder in the isomerization catalyst composition that includes the ZSM-5 zeolite can be relatively low surface area binders, such as alumina and/or silica having a specific area of ≤150 m²/g.

In making the isomerization catalyst composition, the as-synthesized or calcined ZSM-5 zeolite can be mixed with other materials such as the binder, a second zeolite, and/or other components such as water. The mixture can be formed into the desired shape by, e.g., extrusion, molding, and the like. The thus formed catalyst can be optionally dried and/or calcined in nitrogen and/or air to produce the isomerization catalyst composition. It should be understood that the term “extrudate” includes catalysts made via extrusion, molding, or any other process in which the ZSM-5 zeolite is combined with one or more other components such as a binder.

In some embodiments, the isomerization catalyst composition can be an extrudate that can include the ZSM-5 zeolite and a binder, e.g., alumina and/or silica. Such extrudate can include 1 wt % to 99 wt % of the ZSM-5 zeolite and 1 wt % to 99 wt % of the binder, based on the combined weight of the ZSM-5 zeolite and the binder. For example, the extrudate can include 1 wt %, 10 wt %, 20 wt %, 40 wt %, or 50 wt % to 70 wt %, 80 wt %, 90 wt %, 95 wt %, or 99 wt % of the ZSM-5 zeolite and 1 wt %, 5 wt %, 10 wt %, 20 wt %, or 30 wt % to 50 wt %, 60 wt %, 80 wt %, 90 wt %, or 99 wt % of the binder, based on the combined weight of the ZSM-5 zeolite and the binder.

Procedures for preparing silica bound zeolites are described in U.S. Pat. Nos. 4,582,815; 5,053,374; and 5,182,242. A particular procedure for binding ZSM-5 with a silica binder involves an extrusion process. In some embodiments, preparing a silica bound ZSM-5 zeolite can include mixing and extruding a mixture that can include water, ZSM-5 zeolite, colloidal silica, and sodium ions under conditions sufficient to form an uncalcined extrudate having an intermediate green strength sufficient to resist attrition during an ion exchange step. The uncalcined extrudate can be contacted with an aqueous solution that can include ammonium cations under conditions sufficient to exchange cations in the ZSM-5 zeolite with ammonium cations to produce an ammonium exchanged extrudate. The ammonium exchanged extrudate can be calcined under conditions sufficient to generate a hydrogen form of the ZSM-5 zeolite and increase the crush strength of said extrudate.

Another process of silica binding can use a suitable silicone resin, e.g., a high molecular weight, hydroxy functional silicone, such as Dow Corning Q6-2230 silicone resin in a method disclosed in U.S. Pat. No. 4,631,267. Other silicone resins can include those described in U.S. Pat. No. 3,090,691. When a silicone resin is used, a suitable polar, water soluble carrier, such as methanol, ethanol, isopropyl alcohol, N-methyl pyrrolidone or a dibasic ester can also be used along with water as needed. Dibasic esters that may be useful in this invention include dimethyl glutarate, dimethyl succinate, dimethyl adipate, and mixtures thereof.

In some embodiments, extrusion aids can also be used in the preparation of the isomerization catalyst composition. Methyl cellulose is a suitable extrusion aid, and one particular methyl cellulose that can be used can be or can include a hydroxypropyl methyl cellulose, such as K75M METHOCEL®, available from Dow Chemical Co. Methyl cellulose may also be used alone or in combination with other binder or matrix material as a burn-out material to increase the porosity of the isomerization catalyst composition.

In some embodiments, the ZSM-5 zeolite can be at least partially dehydrated prior to contact with the hydrocarbon feed. The ZSM-5 zeolite can be at least partially dehydrated by heating the ZSM-5 zeolite or the isomerization catalyst composition that includes the ZSM-5 zeolite such as the extrudate to a temperature of 100° C., 150° C., or 200° C. to 300° C., 400° C., or 500° C., e.g., 200° C. to 370° C. The ZSM-5 zeolite or catalyst that includes the ZSM-5 zeolite can be heated in a suitable atmosphere such as air, nitrogen, etc. The ZSM-5 zeolite or catalyst that includes the ZSM-5 zeolite can be heated at atmospheric, subatmospheric, or superatmospheric pressure. The ZSM-5 zeolite or catalyst that includes the ZSM-5 zeolite can be heated for 30 minutes, 1 hour, 6 hours, 10 hours, 12 hours, or 18 hours to 20 hours, 24 hours, 30 hours, 36 hours, 42 hours, or 48 hours. Dehydration can also be performed at room temperature merely by placing the ZSM-5 zeolite or the isomerization catalyst composition that includes the ZSM-5 zeolite in a vacuum, but a longer time can be required to obtain a preferred amount of dehydration.

### I.5 The Isomerization Process

In some embodiments, a hydrocarbon feed that includes C8 aromatic hydrocarbons, e.g., meta-xylene and/or ortho-xylene, can be contacted with the isomerization catalyst composition that can be or can include the ZSM-5 zeolite in a conversion zone under conversion zone conditions to effect isomerization of at least a portion of the C8 aromatic hydrocarbons to produce a conversion product rich in para-xylene. The isomerization can be carried out under conditions such that the C8 aromatic hydrocarbons are substantially in the liquid phase in the presence of the isomerization catalyst composition that includes the ZSM-5 zeolite. In some embodiments, an internal pressure in the conversion zone can be sufficient to maintain a majority, e.g., ≥50 mol %, ≥60 wt %, ≥70 wt %, ≥80 mol %, ≥85 mol %, ≥90 mol %, ≥95 mol %, ≥98 mol %, or even substantially all of the C8 aromatic hydrocarbons in the hydrocarbon feed, in the liquid phase at the given temperature in the conversion zone. For example, for a liquid phase isomerization reaction temperature of 240° C., the pressure is typically ≥1,830 kPa absolute.

The hydrocarbon feed and the isomerization catalyst composition can be contacted with one another at a temperature of 140° C., 150° C., 180° C., or 200° C. to 280°, 300° C., 340° C., 370° C., or 400° C. In some embodiments, the hydrocarbon feed and the isomerization catalyst composition can be contacted with one another at a temperature of 140° C. to 400° C., 150° C. to 300° C., or 200° C. to 280° C. The hydrocarbon feed can be contacted with the isomerization catalyst composition at a WHSV of 0.1 hr⁻¹, 0.5 hr⁻¹, 1 hr⁻¹, 5 hr⁻¹, or 10 hr⁻¹ to 12 hr⁻¹, 13 hr⁻¹, 15 hr⁻¹, 16 hr⁻¹, 18 hr⁻¹, or 20 hr⁻¹. In some embodiments, the hydrocarbon feed and can be contacted with the isomerization catalyst composition at a WHSV of 0.1 hr⁻¹ to 20 hr⁻¹, 1 hr⁻¹ to 15 hr⁻¹, or 4 hr⁻¹ to 12 hr⁻¹. In some embodiments, the hydrocarbon feed and the isomerization catalyst composition can be contacted with one another in the presence of molecular hydrogen. The molecular hydrogen can be introduced as a component of the hydrocarbon feed, introduced into the conversion zone, or a combination thereof. The molar ratio of molecular hydrogen to hydrocarbons in the hydrocarbon feed within the conversion zone can be 0.01, 0.05, 0.1, 0.5, 0.7, or 0.8 to 1, 1.3, 1.5, 1.7, or 2. In some embodiments, the hydrocarbon feed and the isomerization catalyst composition can be contacted with one another in the absence of any molecular hydrogen.

As discussed above, an advantage of the process for converting the C8 aromatic hydrocarbons using the isomerization catalyst composition that includes the ZSM-5 zeolite disclosed herein can be a high para-xylene selectivity in the conversion product and in some embodiments at high WHSV such as >10 hr⁻¹. Thus, in some embodiments, the process for converting the C8 aromatic hydrocarbons, as disclosed herein, can exhibit a para-xylene selectivity of ≥19%, ≥20%, ≥21%, ≥22%, ≥23%, or 23.5% at a WHSV of 2.5 hr⁻¹ when the hydrocarbon feed includes para-xylene at a concentration of ≤15 wt %, ≤10 wt %, ≤8 wt %, ≤6 wt %, ≤5 wt %, ≤3 wt %, or ≤2 wt %, based on the total weight of xylenes in the hydrocarbon feed. In other embodiments, the process for converting the C8 aromatic hydrocarbons can exhibit a para-xylene selectivity of ≥19%, ≥20%, or ≥21%, or ≥22%, ≥23%, or ≥23.5% at a WHSV of 5 hr⁻¹ when the hydrocarbon feed includes para-xylene at a concentration of ≤15 wt %, ≤10 wt %, ≤8 wt %, ≤6 wt %, ≤5 wt %, ≤3 wt %, or ≤2 wt %, based on the total weight of xylenes in the hydrocarbon feed. In other embodiments, the process for converting the C8 aromatic hydrocarbons can exhibit a para-xylene selectivity of ≥19%, ≥20%, or ≥21%, or ≥22%, or ≥23% at a WHSV of 10 hr⁻¹ when the C8 hydrocarbon feed includes para-xylene at a concentration of ≤15 wt %, ≤10 wt %, ≤8 wt %, ≤6 wt %, ≤5 wt %, ≤3 wt %, or ≤2 wt %, based on the total weight of xylenes in the C8 hydrocarbon feed. Such high para-xylene selectivity at such a high WHSV is not achievable in a comparative process using a traditional ZSM-5 based catalyst and is particularly advantageous. The fact that the isomerization catalyst composition that includes the ZSM-5 zeolite as disclosed herein can achieve such high para-xylene selectivity at such a high WHSV was surprising and unexpected.

The conversion processes described herein can be carried out as a batch type, semi-continuous, or continuous operation. After use in a moving or fluidized bed reactor, the isomerization catalyst composition(s) can be regenerated in a regeneration zone in which coke is burned from the isomerization catalyst composition(s) in an oxygen containing atmosphere, e.g., air, at an elevated temperature after which the regenerated catalyst can be recycled to the conversion zone, the first conversion zone, or the second conversion zone, depending on the particular process configuration. In a fixed bed reactor, regeneration can be carried out in a conventional manner by using initially an inert gas containing a small amount of oxygen (0.5 vol % to 10 vol %) to burn coke in a controlled manner

In some embodiments, the xylene isomerization reaction can be carried out in a fixed bed reactor. In one embodiment, the isomerization catalyst composition that includes the ZSM-5 zeolite can be disposed in a catalyst bed located within the conversion zone and the hydrocarbon feed can be contacted therewith.

The liquid phase isomerization process is more energy efficient than a vapor phase isomerization process. A vapor phase isomerization process, on the other hand, can be more effective in converting ethylbenzene than a liquid phase isomerization process. Thus, if a hydrocarbon feed subject to isomerization conversion comprises ethylbenzene at an appreciable concentration, it may accumulate in a xylenes loop that includes only a liquid phase isomerization unit without a vapor phase isomerization reactor unit unless a portion of the feed is purged. Purging of the feed or ethylbenzene accumulation in the xylenes loop can both be undesirable. As such, it can be desired to maintain both a liquid phase isomerization unit and a vapor phase isomerization unit in an aromatics production complex. In such case, the liquid phase isomerization and the vapor phase isomerization units can be fed with the hydrocarbon feeds with the same or different compositions with various quantities. In one embodiment, the liquid phase isomerization unit and the vapor phase isomerization unit can be arranged in parallel so that they can receive the aromatic feed from a common source with substantially the same composition. In another embodiment, the liquid phase isomerization unit and the vapor phase isomerization unit can operate in series, such that an hydrocarbon feed is first fed into the liquid phase isomerization unit to accomplish at least a partial isomerization of the xylenes to produce a liquid phase isomerization effluent which, in turn, can be fed into the vapor phase isomerization unit, where additional xylenes isomerization and ethylbenzene conversion can occur. Alternatively, the vapor phase isomerization unit can be the lead unit receiving the hydrocarbon feed and produce an ethylbenzene-depleted vapor phase isomerization effluent which, in turn, can be fed into the liquid phase isomerization unit to further xylene isomerization reactions.

### I.6 The Hydrocarbon Feed

The hydrocarbon feed that includes the C8 aromatic hydrocarbons can be derived from, e.g., an effluent from a C8 aromatic hydrocarbon distillation column, a para-xylene depleted raffinate stream produced from a para-xylene separation/recovery system that includes an adsorption chromatography system, and/or a para-xylene depleted filtrate stream produced from a para-xylene separation/recovery system that includes a para-xylene crystallizer, or a mixture thereof. In this disclosure, the raffinate stream and the filtrate stream are collectively referred to as a raffinate stream below.

The hydrocarbon feed that includes C8 aromatics can include para-xylene at various concentrations. In some embodiments, the hydrocarbon feed can include 0.5 wt %, 1 wt %, 2 wt %, 3 wt %, 4 wt %, 5 wt %, 6 wt %, 7 wt %, 8 wt %, 9 wt %, or 10 wt % to 11 wt %, 12 wt %, 13 wt %, 14 wt %, 15 wt %, 16 wt %, 17 wt %, 18 wt %, 19 wt %, or 20 wt % of para-xylene, based on the total weight of the hydrocarbon feed. Typically, the concentration of para-xylene can be lower than the para-xylene concentration in an equilibrium mixture consisting of para-xylene, meta-xylene, and ortho-xylene at the same temperature. In some embodiments, the concentration of para-xylene in the hydrocarbon feed can be ≤15 wt %, ≤10 wt %, ≤8 wt %, ≤6 wt %, ≤4 wt %, ≤3 wt %, or ≤2 wt %, based on the total weight of the hydrocarbon feed.

The hydrocarbon feed that includes C8 aromatics can include meta-xylene at various concentrations. In some embodiments, the hydrocarbon feed can include 20 wt %, 25 wt %, 30 wt %, 35 wt %, 40 wt %, 45 wt %, or 50 wt % to 55 wt %, 60 wt %, 65 wt %, 70 wt %, 75 wt %, or 80 wt % of meta-xylene, based on the total weight of the hydrocarbon feed. In some embodiments, the concentration of meta-xylene can be significantly higher than the meta-xylene concentration in an equilibrium mixture consisting of para-xylene, meta-xylene, and ortho-xylene at the same temperature, especially if the hydrocarbon feed consists essentially of xylenes only and is substantially free of ethylbenzene.

The hydrocarbon feed that includes C8 aromatics can include ortho-xylene at various concentrations. In some embodiments, the hydrocarbon feed can include 10 wt %, 15 wt %, 20 wt %, or 25 wt % to 30 wt %, 35 wt %, 40 wt %, 45 wt %, or 50 wt % of ortho-xylene, based on the total weight of the hydrocarbon feed. In some embodiments, the concentration of ortho-xylene can be significantly higher than the ortho-xylene concentration in an equilibrium mixture consisting of para-xylene, meta-xylene, and ortho-xylene at the same temperature, especially if the hydrocarbon feed consists essentially of xylenes only and is substantially free of ethylbenzene.

Among all xylenes present in the hydrocarbon feed, meta-xylene and ortho-xylene can be present at any ratio. Thus, the ratio of meta-xylene to ortho-xylene can be 0.1, 0.2, 0.3, 0.4, 0.5, 0.6, 0.7, 0.8, 0.9, or 1 to 2, 3, 4, 5, 6, 7, 8, 9, or 10. In some embodiments the hydrocarbon feed can include xylenes in total at a concentration of 50 wt %, 55 wt %, 60 wt %, 65 wt %, 70 wt %, 75 wt %, or 80 wt % to 85 wt %, 90 wt %, 91 wt %, 92 wt %, 93 wt %, 94 wt %, 95 wt %, 96 wt %, 97 wt %, 98 wt %, 99 wt %, or 100 wt %, based on the total weight of the hydrocarbon feed.

In some embodiments, the hydrocarbon feed can consist essentially of xylenes and ethylbenzene. The hydrocarbon feed can include 1 wt %, 2 wt %, 3 wt %, 4 wt %, 5 wt %, 6 wt %, 7 wt %, 8 wt %, 9 wt %, 10 wt %, 12 wt %, 14 wt %, or 15 wt % to 16 wt %, 18 wt %, 20 wt %, 22 wt %, 24 wt %, 25 wt %, 26 wt %, 28 wt %, or 30 wt % of ethylbenzene, based on the total weight of the hydrocarbon feed. In other embodiments, the hydrocarbon feed can include 2 wt % to 25 wt %, 3 wt % to 20 wt %, or 5 wt % to 15 wt % of ethylbenzene, based on the total weight of the hydrocarbon feed.

In some embodiments, the hydrocarbon feed can include C8 aromatic hydrocarbons, i.e., xylenes and ethylbenzene, at an aggregate concentration of 90 wt %, 92 wt %, 94 wt %, or 95 wt % to 96 w%, 98 wt %, 99 wt %, or 100 wt %, based on the total weight of the hydrocarbon feed. The hydrocarbon feed can also include C9+ aromatic hydrocarbons. In some embodiments, the hydrocarbon feed can include 0.1 wt %, 0.5 wt %, 0.7 wt %, 1 wt %, 3 wt %, or 5 wt % to 10 wt %, 15 wt %, 20 wt %, 25 wt %, or 30 wt % of C9+ aromatic hydrocarbons, based on the total weight of the hydrocarbon feed. In some embodiments, the hydrocarbon feed, depending on its source (e.g., a xylenes distillation column, a para-xylene crystallizer, and/or an adsorption chromatography separation system), can include toluene at various amounts, but typically not greater than 1 wt %, based on the total weight of the hydrocarbon feed. The hydrocarbon feed, depending on its source, can also include C7− aromatic hydrocarbons, e.g., toluene and benzene in total, at various amounts.

### I.7 Recovering a Para-Xylene Product

A high purity para-xylene product can be obtained by separating para-xylene from the conversion product rich in para-xylene that can also include ortho-xylene, meta-xylene, and/or ethylbenzene in a para-xylene separation/recovery system. The para-xylene recovery system can include, e.g., a crystallizer and/or an adsorption chromatography separating system known in the prior art. A para-xylene-depleted product recovered from the para-xylene recovery system (the “filtrate” from a crystallizer upon separation of the para-xylene crystals, or the “raffinate” from the adsorption chromatography separating system, collectively “raffinate”) can be rich in meta-xylene and/or ortho-xylene and include para-xylene at a concentration typically below its concentration in an equilibrium mixture consisting of meta-xylene, ortho-xylene, and para-xylene. To increase the yield of para-xylene, the raffinate stream can be fed into an isomerization unit, where the xylenes can undergo isomerization reactions in contacting the isomerization catalyst composition that includes the ZSM-5 zeolite to produce an isomerized effluent rich in para-xylene compared to the raffinate. At least a portion of the isomerized effluent, after optional separation and removal of lighter hydrocarbons that can be produced in the isomerization unit, can be recycled to the para-xylene recovery system, forming a “xylenes loop.” The recovery of products from a conversion product that includes para-xylene and one or more of: ethylbenzene, meta-xylene, ortho-xylene, benzene, toluene, trimethylbenzenes can include the processes and systems described in U.S. Pat. Nos. 4,899,011; 5,689,027; 5,977,420; and 8,273,934 and WO Publication No.:

02/088056.

### II. The Second Aspect of this Disclosure

The second aspect of this disclosure generally relates to a process for converting a hydrocarbon feed comprising C8 aromatic hydrocarbons, which can comprise one or more of (B-I) providing a precursor catalyst composition exhibiting a first external surface area of a1 m²/g; (B-II) treating the precursor catalyst composition to obtain an isomerization catalyst composition, wherein the isomerization catalyst composition exhibits a second external surface area of a2 m²/g, where (a2−a1)/a1*100%≥10%; (B-III) feeding the hydrocarbon feed into a conversion zone; and (B-IV) contacting the hydrocarbon feed at least partly in a liquid phase with the isomerization catalyst composition in the conversion zone under conversion conditions to effect isomerization of at least a portion of the C8 aromatic hydrocarbons to produce a conversion product rich in para-xylene.

The hydrocarbon feed in the process of the second aspect can be similar to or the same as the hydrocarbon feed as described above in connection with the process of the first aspect. The hydrocarbon feed can comprise, consist essentially of, or consist of, aromatic hydrocarbons. The hydrocarbon feed can comprise, consist essentially of, or consist of, C8 aromatic hydrocarbons. The hydrocarbon feed can comprise, consist essentially of, or consist of, xylenes. In certain embodiments, the hydrocarbon feed can comprise a small quantity (e.g., ≤20wt %, ≤15 wt %, ≤10 wt %, ≤5 wt %, ≤3 wt %, ≤2 wt %, ≤1 wt %, based on the total weight of the hydrocarbon feed) of non-aromatic hydrocarbons. In certain embodiments, the hydrocarbon feed can comprise a small quantity (e.g., ≤20wt %, ≤15 wt %, ≤10 wt %, ≤5 wt %, ≤3 wt %, ≤2 wt %, ≤1 wt %, based on the total weight of the hydrocarbon feed) of ethylbenzene.

The precursor catalyst composition can comprise, consist essentially of, or consist of a catalytically active component. In addition, the precursor catalyst composition can comprise an auxiliary component, such as a co-catalyst, a second catalytically active component, or a catalytically inert component. A non-limiting example of an auxiliary component is a binder or a matrix material. Non-limiting examples of the catalytically active component are the molecular sieves capable of catalyzing an aromatic hydrocarbon isomerization reactions. Such molecular sieves can comprise one or more zeolites. Non-limiting examples of useful zeolites include: ZSM-5, ZSM-11, ZSM-5 and ZSM-11 intergrowth, ZSM-22, ZSM-23, ZSM-35, ZSM-48, a MWW framework zeolite such as MCM-22, MCM-36, MCM-49, MCM-56, PSH-3, SSZ-25, ERB-1, ITQ-1, ITQ-2, UZM-8, UZM-8HS, and mixtures and combinations thereof. ZSM-5 is described in U.S. Pat. Nos. 3,702,886 and Re. 29,948. ZSM-11 is described in U.S. Pat. No. 3,709,979. ZSM-12 is described in U.S. Pat. No. 3,832,449. ZSM-22 is described in U.S. Pat. No. 4,556,477. ZSM-23 is described in U.S. Pat. No. 4,076,842. ZSM-35 is described in U.S. Pat. No. 4,016,245. ZSM-48 is more particularly described in U.S. Pat. No. 4,234,231. MCM-22 is described in U.S. Pat. No. 4,954,325. PSH-3 is described in U.S. Pat. No. 4,439,409. SSZ-25 is described in U.S. Pat. No. 4,826,667. ERB-1 is described in European Patent No. 0293032. ITQ-1 is described in U.S. Pat. No. 6,077,498. ITQ-2 is described in International Patent Publication No. WO97/17290. MCM-36 is described in U.S. Pat. No. 5,250,277. MCM-49 is described in U.S. Pat. No. 5,236,575. MCM-56 is described in U.S. Pat. No. 5,362,697. UZM-8 is described in U.S. Pat. No. 6,756,030. UZM-8HS is described in U.S. Pat. No. 7,713,513. Non-limiting examples of the binder include silica, alumina, zirconia, a titanium oxide, a thorium oxide, yttria, a chromium oxide, a manganese oxide, hafnia, a lanthanide oxide, an alkali metal oxide, an alkaline earth metal oxide, and combinations, mixtures, and compounds thereof.

The precursor catalyst composition can have one or more of the following features: (i) a silica (SiO₂) to alumina (Al₂O₃) molar ratio of r3 to r4, where r3 and r4 can be, independently, e.g., 10, 15, 20, 25, 30, 35, 40, 45, 50, 55, 60, 65, 70, 75, 80, 85, 90, 95, 100, as long as r3<r4; (ii) a total surface area of s(t)3 to s(t)4 m²/g, where s(t)3 and s(t)4 can be, independently, e.g., 200, 250, 300, 350, 400, 450, 500, 550, 600, 650, 700, as long as s(t)3<s(t)4; and (iii) a micropore surface area of s(mp)3 to s(mp)4 m²/g, where s(mp)3 and s(mp)4 can be, independently, e.g., 50, 100, 150, 200, 250, 300, 350, 400, 450, 500, 550, 600. In certain embodiments, including but not limited to those where the precursor catalyst composition comprises a zeolite such as ZSM-5, the precursor catalyst composition may have an external surface area (i.e., a mesopore surface area) of from s(e)3 to s(e)4 m²/g, where s(e)3 and s(e)4 can be, independently, e.g., 10, 15, 20, 25, 30, 35, 40, 45, 50, 55, 60, 65, 70, 75, 80, 85, 90, 95, 100, 110, 120, 130, 140, 150, as long as s(e)3<s(e)4. In certain embodiments s(e)4<55, such as where the precursor catalyst composition comprises ZSM-5 as described in connection with the first aspect of this disclosure.

One treatment method for step (B-II) can comprise: (B-II-1) contacting the precursor catalyst composition with an alkaline aqueous solution; and subsequently (B-II-2) washing and drying the contacted precursor catalyst composition. Non-limiting examples of the useful alkaline aqueous solutions are those comprising LiOH, NaOH, KOH, RbOH, CsOH, Na₂CO₃, Mg(OH)₂, Ca(OH)₂, Sr(OH)₂, and mixtures thereof. Without intending to be bound by a particular theory, it is believed that contacting of such alkaline aqueous solution with the precursor catalyst composition, particularly the catalytically active component therein, can cause the etching and enlargement of a portion of the micropores present in the precursor catalyst composition, resulting in an increase of external surface area (i.e., mesopore surface area) in the treated precursor catalyst composition. In cases where the catalytically active component comprises a molecular sieve such as a zeolite, the alkaline aqueous solution can react with the SiO₂ and/or Al₂O₃ structural component therein to enlarge at least a portion of the micropores therein to mesopores, thereby increasing the measured external surface area of the treated precursor catalyst composition.

Another contemplated approach for step (B-II) can comprise: (B-II-3) contacting the precursor catalyst composition with an aqueous solution of NH₄F·HF; and subsequently (B-II-4) washing and drying the contacted precursor catalyst composition. The acidic NH₄F·HF solution can also etch the micropores present in the precursor catalyst composition to result in an increase of external surface area.

US2013/0183231 A1 discloses processes for introducing mesopores into a zeolitic material to enlarge its external surface area using a combination of acid treatment, surfactant treatment, followed by an alkaline solution treatment, the content of which is incorporated herein by reference in its entirety. The various processes disclosed in US2013/0183231 A1 may be used in step (B-II) to obtain the isomerization catalyst composition from a precursor catalyst composition comprising a zeolite.

Prior to the treatment step (B-II), the precursor catalyst composition exhibits an external surface area of a1 m²/g. The treatment in step (B-II) results in an increased external surface area of the treated precursor catalyst composition of a2 m²/g, where a2>a1. While in general a large increase of external surface area is desirable, in certain embodiments, x1%≤(a2−a1)/a1*100%≤x2%, where x1 and x2, can be, independently, e.g., 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as x1<x2. Preferably x1=30 and x2=800. Preferably x1=40 and x2=500. More preferably x1=50 and x2=300.

An isomerization catalyst composition fabricated by treating a precursor catalyst composition in step (B-II) can exhibit an increased performance at least in terms of para-xylene selectivity in step (B-IV) compared to the precursor catalyst composition under the same isomerization conversion conditions. Thus, in step (B-IV) using the isomerization catalyst composition, a para-xylene selectivity of sel(pX)2 wt % can be obtained. In contrast, in a reference step (B-IV-ref) below, a para-xylene selectivity of sel(pX)1 wt %, where sel(pX)1<sel(pX)2, is obtained: (B-IV-ref) contacting the hydrocarbon feed at least partly in a liquid phase with the precursor catalyst composition in the conversion zone under the same conversion conditions in step (B-IV) to effect isomerization of at least a portion of the C8 aromatic hydrocarbons to produce a reference conversion product rich in para-xylene. Desirably and advantageously,

$$y 1 \% \leq \frac{( sel ( p X ) 2 - sel ( p X ) 1}{sel ( p X ) 1} \times 100 \% \leq y 2 \% ,$$

where y1 and y2 can be, independently, e.g., 3, 4, 5, 6, 7, 8, 9, 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as y1<y2. Without intending to be bound by a particular theory, it is believed that the enlarged external surface in the isomerization catalyst composition improved the catalytic activity compared to the precursor catalyst composition.

In certain embodiments, the isomerization catalyst composition useful in the processes of the second aspect of this disclosure can comprise a zeolite having one or more of the following features: (i) a silica (SiO₂) to alumina (Al₂O₃) molar ratio of r1 to r2, where r1 and r2 can be, independently, e.g., 10, 15, 20, 25, 30, 35, 40, 45, 50, 55, 60, 65, 70, 75, 80, 85, 90, 95, 100, as long as r1<r2; (ii) a total surface area of s(t)1 to s(t)2 m²/g, where s(t)1 and s(t)2 can be, independently, e.g., 200, 250, 300, 350, 400, 450, 500, 550, 600, 650, 700, as long as s(t)1<s(t)2; (iii) a micropore surface area of s(mp)1 to s(mp)2 m²/g, where s(mp)1 and s(mp)2 can be, independently, e.g., 50, 100, 150, 200, 250, 300, 350, 400, 450, 500, 550, 600; and (iv) an external surface area of s(e)1 to s(e)2 m²/g, where s(e)1 and s(e)2 can be, independently, e.g., 55, 60, 65, 70, 75, 80, 85, 90, 95, 100, 120, 140, 150, 160, 180, 200, 220, 240, 250, 260, 280, 300, 350, 400, 450, 500, 550, as long as se1<se2.

Similar to the precursor catalyst composition, in certain embodiments the isomerization catalyst composition useful in the processes of the second aspect of this disclosure can comprise a binder. Such binder can be selected from, e.g., silica, alumina, zirconia, a titanium oxide, a thorium oxide, yttria, a chromium oxide, a manganese oxide, hafnia, a lanthanide oxide, an alkali metal oxide, an alkaline earth metal oxide, and combinations, mixtures, and compounds thereof. In certain embodiments, the binder can be present at an amount of from c(b)1 to c(b)2 wt %, based on the total weight of the isomerization catalyst composition, where c(b)1 and c(b)2 can be, independently, e.g., 1, 2, 3, 4, 5, 6, 7, 8, 9, 10, 15, 20, 25, 30, 35, 40, 45, 50, 55, 60, 65, 70, 75, 80, 85, 90, 95, as long as c(b)1<c(b)2. In certain embodiments where the precursor catalyst composition is free of a binder, the isomerization catalyst composition may be free of a binder as well. For example, the isomerization catalyst composition can consist essentially of or consist of one or more molecular sieves, e.g., one or more zeolites, such as one or more of ZSM-5, ZSM-11, ZSM-5 and ZSM-11 intergrowth, ZSM-22, ZSM-23, ZSM-48, a MWW framework zeolite such as MCM-22, MCM-36, MCM-49, MCM-56, and mixtures and combinations thereof.

The isomerization catalyst composition can take any form of a catalyst composition suitable for the contacting step (B-IV). Non-limiting examples of the forms of the isomerization catalyst composition include: powder; pellets; slurry; extrudates; and the like, of any suitable geometric shape and size. A particularly desirable form is extrudate. In step (B-IV), the isomerization catalyst composition may be present in the conversion zone in a fixed bed, a moving bed, a slurry, and the like, suitable for the conversion reactions under the conversion conditions. In certain embodiments, the conversion conditions can comprise the conversion conditions comprises at least one of the following: (i) a temperature in a range from T1 to T2° C., where T1 and T2 can be, independently, e.g., 160, 170, 180, 190, 200, 210, 220, 230, 240, 250, 260, 270, 280, 300, 350, as long as T1<T2. A great advantage of the LPI process of the second aspect of this disclosure is the lower temperature in the conversion zone compared to vapor phase only isomerization of C8 aromatic hydrocarbons. The lower LPI temperature translates to higher energy efficiency; (ii) an absolute pressure in a range from p1 to p2 kilopascal, where p1 and p2 can be, independently, e.g., 100, 200, 300, 400, 500, 600, 700, 800, 900, 1000, 1500, 2000, 3000, 4000, 5000, as long as p1<p2; (iii) a molecular hydrogen (H₂) concentration in the hydrocarbon feed in a range from c(H₂)1 to c(H₂)2 ppm by weight, based on the total weight of the hydrocarbon feed, where c(H₂)1 and c(H₂)2 can be, independently, e.g., 0, 1, 3, 4, 5, 6, 7, 8, 9, 10, 15, 20, 25, 30, 40, 50, 60, 70, 80, 90, 100, 150, 200, 250, 300, 350, 400, 450, 500, as long as c(H₂)1<c(H₂)2; and (iv) a WHSV of the hydrocarbon feed in a range from w1 to w2 hr−1, where w1 and w2 can be, independently, e.g., 0.5, 1, 1.5, 2, 2.5, 3, 3.5, 4, 5, 6, 7, 8, 9, 10, 11, 12, 13, 14, 15, 16, 17, 18, 19, 20, as long as w1<w2. Preferably c(H₂)2≤200. Preferably c(H₂)2≤100. Preferably c(H₂)2≤50. Preferably c(H₂)2≤10. Preferably no H₂ is cofed into the conversion zone. At low H₂ concentration, the H₂ can be completely dissolved in the liquid phase in hydrocarbon feed, which is highly advantageous. Conventional vapor-phase-only isomerization process typically requires the presence of H₂ at a higher feeding rate, which leads to more complex reactor design and subsequent separation

### III. The Third Aspect of this Disclosure

The third aspect of this disclosure generally relates to a process for converting a hydrocarbon feed comprising C8 aromatic hydrocarbons, which can comprise one or more of (C-I) providing a precursor catalyst composition exhibiting a first external surface area of a1 m²/g; (C-II) treating the precursor catalyst composition to obtain a treated precursor catalyst composition, wherein the treated precursor catalyst composition exhibits a second external surface area of a2 m²/g, wherein (a2−a1)/a1*100%≥10%; (C-III) forming an isomerization catalyst composition from the treated precursor catalyst composition; (C-IV) feeding the hydrocarbon feed into a conversion zone; and (C-V) contacting the hydrocarbon feed at least partly in a liquid phase with the isomerization catalyst composition in the conversion zone under conversion conditions to effect isomerization of at least a portion of the C8 aromatic hydrocarbons to produce a conversion product rich in para-xylene.

The hydrocarbon feed in the process of the third aspect can be similar to or the same as the hydrocarbon feed as described above in connection with the processes of the first and/or second aspects. The hydrocarbon feed can comprise, consist essentially of, or consist of, aromatic hydrocarbons. The hydrocarbon feed can comprise, consist essentially of, or consist of, C8 aromatic hydrocarbons. The hydrocarbon feed can comprise, consist essentially of, or consist of, xylenes. In certain embodiments, the hydrocarbon feed can comprise a small quantity (e.g., ≤20wt %, ≤15 wt %, ≤10 wt %, ≤5 wt %, ≤3 wt %, ≤2 wt %, ≤1 wt %, based on the total weight of the hydrocarbon feed) of non-aromatic hydrocarbons. In certain embodiments, the hydrocarbon feed can comprise a small quantity (e.g., ≤20wt %, ≤15 wt %, ≤10 wt %, ≤5 wt %, ≤3 wt %, ≤2 wt %, ≤1 wt %, based on the total weight of the hydrocarbon feed) of ethylbenzene.

The precursor catalyst composition can comprise, consist essentially of, or consist of a catalytically active component. In addition, the precursor catalyst composition can comprise an auxiliary component, such as a co-catalyst, a second catalytically active component, or a catalytically inert component. A non-limiting example of an auxiliary component is a binder or a matrix material. Non-limiting examples of the catalytically active component are the molecular sieves capable of catalyzing an aromatic hydrocarbon isomerization reaction. Such molecular sieves can comprise one or more zeolites. Non-limiting examples of useful zeolites include: ZSM-5, ZSM-11, ZSM-5 and ZSM-11 intergrowth, ZSM-22, ZSM-23, ZSM-48, a MWW framework zeolite such as MCM-22, MCM-36, MCM-49, MCM-56, and mixtures and combinations thereof. Non-limiting examples of the binder include silica, alumina, zirconia, a titanium oxide, a thorium oxide, yttria, a chromium oxide, a manganese oxide, hafnia, a lanthanide oxide, an alkali metal oxide, an alkaline earth metal oxide, and combinations, mixtures, and compounds thereof. In preferred embodiments, the precursor catalyst composition consists essentially of or consist of one or more zeolites, such as those listed earlier in this paragraph. In a particularly preferred embodiment, the precursor catalyst composition consists essentially of or consist of ZSM-5, such as an as-synthesized ZSM-5, particularly one having an external surface area of <55 m²/g.

The precursor catalyst composition can have one or more of the following features: (i) a silica (SiO₂) to alumina (Al₂O₃) molar ratio of r3 to r4, where r3 and r4 can be, independently, e.g., 10, 15, 20, 25, 30, 35, 40, 45, 50, 55, 60, 65, 70, 75, 80, 85, 90, 95, 100, as long as r3<r4; (ii) a total surface area of s(t)3 to s(t)4 m²/g, where s(t)3 and s(t)4 can be, independently, e.g., 200, 250, 300, 350, 400, 450, 500, 550, 600, 650, 700, as long as s(t)3<s(t)4; and (iii) a micropore surface area of s(mp)3 to s(mp)4 m²/g, where s(mp)3 and s(mp)4 can be, independently, e.g., 50, 100, 150, 200, 250, 300, 350, 400, 450, 500, 550, 600. In certain embodiments, including but not limited to those where the precursor catalyst composition comprises a zeolite such as ZSM-5, the precursor catalyst composition may have an external surface area (i.e., a mesopore surface area) of from s(e)3 to s(e)4 m²/g, where s(e)3 and s(e)4 can be, independently, e.g., 10, 15, 20, 25, 30, 35, 40, 45, 50, 55, 60, 65, 70, 75, 80, 85, 90, 95, 100, 110, 120, 130, 140, 150, as long as s(e)3<s(e)4. In certain embodiments s(e)4<55, such as where the precursor catalyst composition comprises ZSM-5 as described in connection with the first aspect of this disclosure.

One treatment method for step (C-II) can comprise: (C-II-1) contacting the precursor catalyst composition with an alkaline aqueous solution; and subsequently (C-II-2) washing and drying the contacted precursor catalyst composition. Non-limiting examples of the useful alkaline aqueous solutions are those comprising LiOH, NaOH, KOH, RbOH, CsOH, Na₂CO₃, Mg(OH)₂, Ca(OH)₂, Sr(OH)₂, and mixtures thereof. Without intending to be bound by a particular theory, it is believed that contacting of such alkaline aqueous solution with the precursor catalyst composition, particularly the catalytically active component therein, can cause the etching and enlargement of a portion of the micropores present in the precursor catalyst composition, resulting in an increase of external surface area (i.e., mesopore surface area) in the treated precursor catalyst composition. In cases where the catalytically active component comprises a molecular sieve such as a zeolite, the alkaline aqueous solution can react with the SiO₂ and/or Al₂O₃ structural component therein to enlarge at least a portion of the micropores therein to mesopores, thereby increasing the measured external surface area of the treated precursor catalyst composition.

Another contemplated approach for step (C-II) can comprise: (C-II-3) contacting the precursor catalyst composition with an aqueous solution of NH₄F·HF; and subsequently (C-II-4) washing and drying the contacted precursor catalyst composition. The acidic NH₄F·HF solution can also etch the micropores present in the precursor catalyst composition to result in an increase of external surface area.

US2013/0183231 A1 discloses processes for introducing mesopores into a zeolitic material to enlarge its external surface area using a combination of acid treatment, surfactant treatment, followed by an alkaline solution treatment, the content of which is incorporated herein by reference in its entirety. The various processes disclosed in US2013/0183231 A1 may be used in step (C-II) to obtain the isomerization catalyst composition from a precursor catalyst composition comprising a zeolite.

Prior to the treatment step (C-II), the precursor catalyst composition exhibits an external surface area of a1 m²/g. The treatment in step (C-II) results in an increased external surface area of the treated precursor catalyst composition of a2 m²/g, where a2>a1. While in general a large increase of external surface area is desirable, in certain embodiments, x1%≤(a2−a1)/a1*100%≤x2%, where x1 and x2, can be, independently, e.g., 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as x1<x2. Preferably x1=30 and x2=800. Preferably x1=40 and x2=500. More preferably x1=50 and x2=300.

In step (C-III) of the processes of the third disclosure, an isomerization catalyst composition is formed from the treated precursor catalyst composition obtained from step (C-II). In certain embodiments, (C-III) can comprise (C-III-1) combining the treated precursor catalyst composition with an auxiliary component; and (C-III-2) obtaining the isomerization catalyst composition from the combined mixture from step (C-III-1). The auxiliary component can include one or more of a co-catalyst, a second catalytically active component different from the catalytic component in the treated precursor catalyst composition, or a catalytically inert component. Non-limiting examples of the second catalytically active component are the molecular sieves capable of catalyzing an aromatic hydrocarbon isomerization reactions. Such molecular sieves can comprise one or more zeolites. Non-limiting examples of useful zeolites include: ZSM-5, ZSM-11, ZSM-5 and ZSM-11 intergrowth, ZSM-22, ZSM-23, ZSM-48, a MWW framework zeolite such as MCM-22, MCM-36, MCM-49, MCM-56, and mixtures and combinations thereof. A non-limiting example of an auxiliary component is a binder or a matrix material. Non-limiting examples of the binder include silica, alumina, zirconia, a titanium oxide, a thorium oxide, yttria, a chromium oxide, a manganese oxide, hafnia, a lanthanide oxide, an alkali metal oxide, an alkaline earth metal oxide, and combinations, mixtures, and compounds thereof. In step (C-III-2), the combined mixture can be formed into any desired geometry and/or size, in such non-limiting forms as powder, pellets, extrudates, and the like. Optionally, a drying and/or calcination step may be carried out to the formed combined mixture to produce the isomerization catalyst composition.

An isomerization catalyst composition fabricated by treating a precursor catalyst composition in step (C-II) and forming in step (C-III) can exhibit an increased performance at least in terms of para-xylene selectivity in step (C-V) compared to the precursor catalyst composition under the same isomerization conversion conditions. Thus, in step (C-V) using the isomerization catalyst composition, a para-xylene selectivity of sel(pX)2 wt % can be obtained. In contrast, in a reference step (C-V-ref) below, a para-xylene selectivity of sel(pX)1 wt %, where sel(pX)1<sel(pX)2, is obtained: (C-V-ref) contacting the hydrocarbon feed at least partly in a liquid phase with the precursor catalyst composition in the conversion zone under the same conversion conditions in step (C-V) to effect isomerization of at least a portion of the C8 aromatic hydrocarbons to produce a reference conversion product rich in para-xylene. Desirably and advantageously,

$$y 1 \% \leq \frac{( sel ( p X ) 2 - sel ( p X ) 1}{sel ( p X ) 1} \times 100 \% \leq y 2 \% ,$$

where y1 and y2 can be, independently, e.g., 3, 4, 5, 6, 7, 8, 9, 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as y1<y2. Without intending to be bound by a particular theory, it is believed that the enlarged external surface in the isomerization catalyst composition improved the catalytic activity compared to the precursor catalyst composition.

In certain embodiments, the isomerization catalyst composition useful in the processes of the third aspect of this disclosure can comprise a zeolite having one or more of the following features: (i) a silica (SiO₂) to alumina (Al₂O₃) molar ratio of r1 to r2, where r1 and r2 can be, independently, e.g., 10, 15, 20, 25, 30, 35, 40, 45, 50, 55, 60, 65, 70, 75, 80, 85, 90, 95, 100, as long as r1<r2; (ii) a total surface area of s(t)1 to s(t)2 m²/g, where s(t)1 and s(t)2 can be, independently, e.g., 200, 250, 300, 350, 400, 450, 500, 550, 600, 650, 700, as long as s(t)1<s(t)2; (iii) a micropore surface area of s(mp)1 to s(mp)2 m²/g, where s(mp)1 and s(mp)2 can be, independently, e.g., 50, 100, 150, 200, 250, 300, 350, 400, 450, 500, 550, 600; and (iv) an external surface area of s(e)1 to s(e)2 m²/g, where s(e)1 and s(e)2 can be, independently, e.g., 55, 60, 65, 70, 75, 80, 85, 90, 95, 100, 120, 140, 150, 160, 180, 200, 220, 240, 250, 260, 280, 300, 350, 400, 450, 500, 550, as long as se1<se2.

Similar to the precursor catalyst composition, in certain embodiments the isomerization catalyst composition useful in the processes of the third aspect of this disclosure can comprise a binder. Such binder can be selected from, e.g., silica, alumina, zirconia, a titanium oxide, a thorium oxide, yttria, a chromium oxide, a manganese oxide, hafnia, a lanthanide oxide, an alkali metal oxide, an alkaline earth metal oxide, and combinations, mixtures, and compounds thereof. In certain embodiments, the binder can be present at an amount of from c(b)1 to c(b)2 wt %, based on the total weight of the isomerization catalyst composition, where c(b)1 and c(b)2 can be, independently, e.g., 1, 2, 3, 4, 5, 6, 7, 8, 9, 10, 15, 20, 25, 30, 35, 40, 45, 50, 55, 60, 65, 70, 75, 80, 85, 90, 95, as long as c(b)1<c(b)2.

The isomerization catalyst composition can take any form of a catalyst composition suitable for the contacting step (C-V). Non-limiting examples of the forms of the isomerization catalyst composition include: powder; pellets; slurry; extrudates; and the like, of any suitable geometric shape and size. A particularly desirable form is extrudate. In step (C-V), the isomerization catalyst composition may be present in the conversion zone in a fixed bed, a moving bed, a slurry, and the like, suitable for the conversion reactions under the conversion conditions. In certain embodiments, the conversion conditions can comprise at least one of the following: (i) a temperature in a range from T1 to T2° C., where T1 and T2 can be, independently, e.g., 160, 170, 180, 190, 200, 210, 220, 230, 240, 250, 260, 270, 280, 300, 350, as long as T1<T2. A great advantage of the LPI process of the third aspect of this disclosure is the lower temperature in the conversion zone compared to vapor phase only isomerization of C8 aromatic hydrocarbons. The lower LPI temperature translates to higher energy efficiency; (ii) an absolute pressure in a range from p1 to p2 kilopascal, where p1 and p2 can be, independently, e.g., 100, 200, 300, 400, 500, 600, 700, 800, 900, 1000, 1500, 2000, 3000, 4000, 5000, as long as p1<p2; (iii) a molecular hydrogen (H₂) concentration in the hydrocarbon feed in a range from c(H₂)1 to c(H₂)2 ppm by weight, based on the total weight of the hydrocarbon feed, where c(H₂)1 and c(H₂)2 can be, independently, e.g., 0, 1, 3, 4, 5, 6, 7, 8, 9, 10, 15, 20, 25, 30, 40, 50, 60, 70, 80, 90, 100, 150, 200, 250, 300, 350, 400, 450, 500, as long as c(H₂)1<c(H₂)2. Preferably c(H₂)2≤200. Preferably c(H₂)2≤100. Preferably c(H₂)2≤50. Preferably c(H₂)2≤10. Preferably no H₂ is cofed into the conversion zone. At low H₂ concentration, the H₂ can be completely dissolved in the liquid phase in hydrocarbon feed, which is highly advantageous. Conventional vapor-phase-only isomerization process typically requires the presence of H2 at a higher feeding rate, which leads to more complex reactor design and subsequent separation; and (iv) a WHSV of the hydrocarbon feed in a range from w1 to w2 hr⁻¹, where w1 and w2 can be, independently, e.g., 0.5, 1, 1.5, 2, 2.5, 3, 3.5, 4, 5, 6, 7, 8, 9, 10, 11, 12, 13, 14, 15, 16, 17, 18, 19, 20, as long as w1<w2.

### EXAMPLES

The foregoing discussion can be further described with reference to the following non-limiting examples.

An inventive isomerization catalyst composition, prepared from a precursor catalyst composition consisting of a ZSM-5 zeolite, and the precursor catalyst composition (aka “parent ZSM-5 zeolite”) were tested in a single-bed system in an identical configuration in Example 1 (Ex. 1) and Comparative Example 1 (CEx. 1), respectively. The inventive isomerization catalyst composition was prepared by treating the precursor catalyst composition by NaOH water solution treatment as described above. The silica to alumina molar ratio, total surface area, micropore surface area, and external surface area (mesopore surface area) of the isomerization catalyst composition and the precursor catalyst composition are reported in the TABLE below. As can be seen, the inventive isomerization catalyst composition exhibited a significantly higher (134% higher) external surface area than the precursor catalyst composition, as a result of the alkaline treatment. Both the precursor catalyst composition and the inventive isomerization catalyst composition as tested in these examples were free of a binder. It is believed that a formulated catalyst composition comprising, in addition to the parent ZSM-5 zeolite or the treated ZSM-5 zeolite as tested in these examples, a binder such as Al₂O₃, SiO₂, ZrO₂, mixtures or combinations or compounds thereof, and the like, in a form such as an extrudate, would have similar catalyst performances.

The hydrocarbon feed used in examples CEx. 1 and Ex. 1 comprised about 13 wt % of ethylbenzene, about 1.5 wt % of C8-C9 non-aromatics, about 1.5 wt % of para-xylene, about 19 wt % of ortho-xylene, and about 66 wt % of meta-xylene.

In both examples, 0.8 gram sample of the catalyst composition was packed in a tubular reactor. To remove moisture, the catalyst composition was dried under flowing nitrogen gas, ramping at 2° C. per minute from room temperature to 240° C. and held at 240° C. for one hour. The isomerization conditions were set to a temperature of 240° C. and a pressure of 1.82 MPag, while the WHSV was varied from 2.5 hr⁻¹ to 10 hr⁻¹. No molecular hydrogen was added during the process. The process conditions and isomerization results are shown in the TABLE below.

| | | TABLE | | | | | |
|---|---|---|---|---|---|---|---|
| | | Example | | | | | |
| | | CEx. 1 | | | Ex. 1 | | |
| Catalyst SiO₂/Al₂O₃ Ratio | | 79 | | | 30.4 | | |
| Catalyst | Total | 443 | | | 471 | | |
| Surface | Micropore | 390 | | | 347 | | |
| Areas (m²/g) | External | 53 | | | 124 | | |
| Testing | Pressure (MPag) | 1.82 | 1.82 | 1.82 | 1.82 | 1.82 | 1.82 |
| Conditions | WHSV (hr⁻¹) | 2.5 | 5 | 10 | 2.5 | 5 | 10 |
| and Results | Temperature (° C.) | 240 | 240 | 240 | 240 | 240 | 240 |
| | EB Conversion (%) | 0.7 | 0.5 | 0.3 | 1.3 | 0.9 | 0.6 |
| | para-Xylene Selectivity (wt %) | 15.2 | 10.3 | 6.7 | 23.7 | 22.8 | 19.6 |

As can be seen from the TABLE, at WHSV of 2.5, 5, and 10 hr⁻¹, Ex. 1 exhibited dramatic increases of para-xylene selectivity, of 55.9%, 121%, and 192%, respectively, relative to the para-xylene selectivity in CEx. 1. Such surprising and unexpected high increases demonstrated the substantially advantageous effect of the higher mesoporous surface area of the inventive isomerization catalyst composition compared to the precursor catalyst composition.

### Listing of Embodiments

This disclosure may further include the following non-limiting embodiments.

A1. A process for converting a hydrocarbon feed comprising C8 aromatic hydrocarbons, the process comprising: (I) feeding the hydrocarbon feed into a conversion zone; and (II) contacting the hydrocarbon feed at least partly in a liquid phase with an isomerization catalyst composition in the conversion zone under conversion conditions to effect isomerization of at least a portion of the C8 aromatic hydrocarbons to produce a conversion product rich in para-xylene, wherein the isomerization catalyst composition comprises a zeolite having a silica (SiO₂) to alumina (Al₂O₃) molar ratio of 10 to 100 , a total surface area of 200 m²/g to 700 m²/g, a micropore surface area of 50 m²/g to 600 m²/g, and an external surface area of 55 m²/g to 550 m²/g, wherein the zeolite can be preferably a ZSM-5 zeolite.

A2. The process of A1, wherein the isomerization catalyst composition is an extrudate comprising the ZSM-5 zeolite and a binder, the binder preferably selected from alumina, silica, zirconia, titania, zircon, a chromium oxide, a combination thereof, or a mixture thereof.

A3. The process of A1 or A2, wherein the silica (SiO₂) to alumina (Al₂O₃) molar ratio is 15 to 60, preferably from 20 to 40.

A4. The process of any of A1 to A3, wherein the total surface area is 300 m²/g to 600 m²/g (preferably 400 m²/g to 500 m²/g), the micropore surface area is 200 m²/g to 550 m²/g (preferably 300 m²/g to 450 m²/g), and the external surface area is 60 m²/g to 350 m²/g (preferably 100 m²/g to 200 m²/g).

A5. The process of any of A1 to A4, wherein the isomerization catalyst composition is an extrudate comprising the ZSM-5 zeolite and a binder.

A6. The process of any of A1 to A5, wherein the silica (SiO₂) to alumina (Al₂O₃) molar ratio is 15 to 60, and the external surface area is 80 m²/g to 350 m²/g.

A7. The process of any of A1 to A6, wherein the silica (SiO₂) to alumina (Al₂O₃) molar ratio is 20 to 40, and the external surface area is 100 m²/g to 200 m²/g.

A8. The process of any of A1 to A7, wherein the ZSM-5 zeolite is in the form of a ZSM-5/ZSM-11 intergrowth zeolite.

A9. The process of any of A1 to A8, wherein the isomerization catalyst composition comprises from 1 wt % to 100 wt % of the ZSM-5 zeolite, based on a total weight of all zeolites present in the isomerization catalyst composition.

A10. The process of any of A1 to A9, wherein: the isomerization catalyst composition is an extrudate comprising the ZSM-5 zeolite and a binder; the binder comprises silica, alumina, or a mixture thereof, and the extrudate comprises 10 wt % to 90 wt % of the binder, based on the combined weight of the ZSM-5 zeolite and the binder.

A11. The process of any of A1 to A10, wherein the conversion conditions comprise an absolute pressure sufficient to maintain the C8 aromatic hydrocarbons in liquid phase, and wherein the conversion conditions comprise a weight hour space velocity of 0.1 hr⁻¹ to 20 hr⁻¹ and a temperature of 140° C. to 400° C.

A12. The process of any of A1 to A11, wherein the conversion conditions comprise an absolute pressure sufficient to maintain the C8 aromatic hydrocarbons in liquid phase, and wherein the conversion conditions comprise a weight hour space velocity of 4 hr⁻¹ to 12 hr⁻¹ and a temperature of 200° C. to 280° C.

A13. The process of any of A1 to A12, wherein molecular hydrogen is fed into the conversion zone, and wherein the molecular hydrogen is present in an amount of 4 ppm to 250 ppm, based on the weight of the hydrocarbon feed.

A14. The process of any of A1 to A12, wherein molecular hydrogen is not fed into the conversion zone.

A15. The process of any of A1 to A14, wherein the hydrocarbon feed comprises ethylbenzene and at least one of ortho-xylene and meta-xylene.

A16. The process of any of A1 to A15, wherein the conversion conditions comprise an absolute pressure sufficient to maintain the C8 aromatic hydrocarbons in liquid phase, and wherein, when the hydrocarbon feed comprises less than 5 wt % of para-xylene, the process exhibits a para-xylene selectivity of at least 16% at a weight hour space velocity of 2.5 hr⁻¹, 5 hr⁻¹, and 10 hr⁻¹.

B1. A process for converting a hydrocarbon feed comprising C8 aromatic hydrocarbons, the process comprising: (B-I) providing a precursor catalyst composition exhibiting a first external surface area of a1 m²/g; (B-II) treating the precursor catalyst composition to obtain an isomerization catalyst composition, wherein the isomerization catalyst composition exhibits a second external surface area of a2 m²/g, wherein (a2−a1)/a1*100%≥10%; (B-III) feeding the hydrocarbon feed into a conversion zone; and (B-IV) contacting the hydrocarbon feed at least partly in a liquid phase with the isomerization catalyst composition in the conversion zone under conversion conditions to effect isomerization of at least a portion of the C8 aromatic hydrocarbons to produce a conversion product rich in para-xylene.

B2. The process of B1, wherein x1%≤(a2−a1)/a1*100%≤x2%, where x1 and x2, can be, independently, e.g., 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as x1<x2.

B3. The process of B1 or B2, wherein step (B-IV) exhibits a para-xylene selectivity of sel(pX)2 wt %, and a reference step (B-IV-ref) below exhibits a para-xylene selectivity of sel(pX)1 wt %: (B-IV-ref) contacting the hydrocarbon feed at least partly in a liquid phase with the precursor catalyst composition in the conversion zone under the same conversion conditions in step (B-IV) to effect isomerization of at least a portion of the C8 aromatic hydrocarbons to produce a reference conversion product rich in para-xylene; where

$$y 1 \% \leq \frac{( sel ( p X ) 2 - sel ( p X ) 1}{sel ( p X ) 1} \times 100 \% \leq y 2 \% ,$$

where y1 and y2, can be, independently, e.g., 3, 4, 5, 6, 7, 8, 9, 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as y1<y2.

B4. The process of any of B1 to B3, wherein step (B-II) comprises: (B-II-1) contacting the precursor catalyst composition with an alkaline aqueous solution; and subsequently (B-II-2) washing and drying the contacted precursor catalyst composition.

B5. The process of B4, wherein the alkaline aqueous solution comprises LiOH, NaOH, KOH, RbOH, CsOH, Na₂CO₃, Mg(OH)₂, Ca(OH)₂, Sr(OH)₂, and mixtures thereof.

B6. The process of any of B1 to B3, wherein step (B-II) comprises: (B-II-3) contacting the precursor catalyst composition with an aqueous solution of NH₄F·HF; and subsequently (B-II-4) washing and drying the contacted precursor catalyst composition.

B7. The process of any of B1 to B6, wherein the precursor catalyst composition comprises a zeolite.

B8. The process of B7, wherein the zeolite comprises one or more of ZSM-5, ZSM-11, ZSM-5 and ZSM-11 intergrowth, ZSM-22, ZSM-23, ZSM-48, a MWW framework zeolite such as MCM-22, 36, 49, 56, and mixtures and combinations thereof.

B9. The process of B7 or B8, wherein the precursor catalyst composition comprises a zeolite having a silica (SiO₂) to alumina (Al₂O₃) molar ratio of 10 to 100, a total surface area of 200 m²/g to 700 m²/g, a micropore surface area of 50 m²/g to 600 m²/g.

B10. The process of any of B1 to B9, wherein the precursor catalyst composition exhibits an external surface area of less than 55 m²/g.

B11. The process of any of B1 to B10, wherein the isomerization catalyst composition comprises a zeolite having one or more of the following features: a silica (SiO₂) to alumina (Al₂O₃) molar ratio of r1 to r2, where r1 and r2 can be, independently, e.g., 10, 15, 20, 25, 30, 35, 40, 45, 50, 55, 60, 65, 70, 75, 80, 85, 90, 95, 100, as long as r1<r2; a total surface area of s(t)1 to s(t)2 m²/g, where s(t)1 and s(t)2 can be, independently, e.g., 200, 250, 300, 350, 400, 450, 500, 550, 600, 650, 700, as long as s(t)1<s(t)2; a micropore surface area of s(mp)1 to s(mp)2 m²/g, where s(mp)1 and s(mp)2 can be, independently, e.g., 50, 100, 150, 200, 250, 300, 350, 400, 450, 500, 550, 600; and an external surface area of s(e)1 to s(e)2 m²/g, where s(e)1 and s(e)2 can be, independently, e.g., 55, 60, 65, 70, 75, 80, 85, 90, 95, 100, 120, 140, 150, 160, 180, 200, 220, 240, 250, 260, 280, 300, 350, 400, 450, 500, 550, as long as se1<se2.

B12. The process of any of B1 to B11, wherein the precursor catalyst composition comprises a binder.

B13. The process of B12, wherein the isomerization catalyst composition comprises the binder.

B14. The process of B12 or B13, wherein the binder is selected from silica, alumina, zirconia, a titanium oxide, a thorium oxide, yttria, a chromium oxide, a manganese oxide, hafnia, a lanthanide oxide, an alkali metal oxide, an alkaline earth metal oxide, and combinations, mixtures, and compounds thereof.

B15. The process of any of B12 to B14, wherein the isomerization catalyst composition comprises the binder at an amount of from c(b)1 to c(b)2 wt %, based on the total weight of the isomerization catalyst composition, where c(b)1 and c(b)2 can be, independently, e.g., 1, 2, 3, 4, 5, 6, 7, 8, 9, 10, 15, 20, 25, 30, 35, 40, 45, 50, 55, 60, 65, 70, 75, 80, 85, 90, 95, as long as c(b)1<c(b)2.

B16. The process of any of B1 to B15, wherein the conversion conditions comprises at least one of the following: (i) a temperature in a range from T1 to T2° C., where T1 and T2 can be, independently, e.g., 160, 170, 180, 190, 200, 210, 220, 230, 240, 250, 260, 270, 280, 300, 350, as long as T1<T2; (ii) an absolute pressure in a range from p1 to p2 kilopascal, where p1 and p2 can be, independently, e.g., 100, 200, 300, 400, 500, 600, 700, 800, 900, 1000, 1500, 2000, 3000, 4000, 5000, as long as p1<p2; (iii) a H2 concentration in the hydrocarbon feed in a range from c(H2)1 to c(H2)2 ppm by weight, based on the total weight of the hydrocarbon feed, where c(H2)1 and c(H2)2 can be, independently, e.g., 0, 1, 3, 4, 5, 6, 7, 8, 9, 10, 15, 20, 25, 30, 40, 50, 60, 70, 80, 90, 100, 150, 200, 250, 300, 350, 400, 450, 500, as long as c(H2)1<c(H2)2; and (iv) a WHSV of the hydrocarbon feed in a range from w1 to w2 hr⁻¹, where w1 and w2 can be, independently, e.g., 0.5, 1, 1.5, 2, 2.5, 3, 3.5, 4, 5, 6, 7, 8, 9, 10, 11, 12, 13, 14, 15, 16, 17, 18, 19, 20, as long as w1<w2.

B17. The process of any of B1 to B16, wherein the precursor catalyst composition is an extrudate.

C1. A process for converting a hydrocarbon feed comprising C8 aromatic hydrocarbons, the process comprising: (C-I) providing a precursor catalyst composition exhibiting a first external surface area of a1 m²/g; (C-II) treating the precursor catalyst composition to obtain a treated precursor catalyst composition, wherein the treated precursor catalyst composition exhibits a second external surface area of a2 m²/g, wherein (a2−a1)/a1*100%≥10%; (C-III) forming an isomerization catalyst composition from the treated precursor catalyst composition; (C-IV) feeding the hydrocarbon feed into a conversion zone; and (C-V) contacting the hydrocarbon feed at least partly in a liquid phase with the isomerization catalyst composition in the conversion zone under conversion conditions to effect isomerization of at least a portion of the C8 aromatic hydrocarbons to produce a conversion product rich in para-xylene.

C2. The process of C1, wherein x1%≤(a2−a1)/a1*100%≤x2%, where x1 and x2, can be, independently, e.g., 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as x1<x2.

C3. The process of C1 or C2, wherein step (C-V) exhibits a para-xylene selectivity of sel(pX)2 wt %, and a reference step (C-V-ref) below exhibits a para-xylene selectivity of sel(pX)1 wt %: (C-V-ref) contacting the hydrocarbon feed at least partly in a liquid phase with the precursor catalyst composition in the conversion zone under the same conversion conditions in step (C-V) to effect isomerization of at least a portion of the C8 aromatic hydrocarbons to produce a reference conversion product rich in para-xylene; where

$$y 1 \% \leq \frac{( sel ( p X ) 2 - sel ( p X ) 1}{sel ( p X ) 1} \times 100 \% \leq y 2 \% ,$$

where y1 and y2, can be, independently, e.g., 5, 10, 20, 30, 40, 50, 60, 70, 80, 90, 100, 120, 140, 150, 160, 180, 200, 220, 240, 250, 260, 280, 300, 320, 340, 350, 360, 380, 400, 420, 440, 450, 460, 480, 500, 600, 700, 800, 900, 1000, as long as y1<y2.

C4. The process of any of C1 to C3, wherein step (C-II) comprises: (C-II-1) contacting the precursor catalyst composition with an alkaline aqueous solution; and subsequently (C-II-2) washing and drying the contacted precursor catalyst composition.

C5. The process of C4, wherein the alkaline aqueous solution comprises LiOH, NaOH, KOH, RbOH, CsOH, Na₂CO₃, Mg(OH)₂, Ca(OH)₂, Sr(OH)₂, and mixtures thereof.

C6. The process of any of C1 to C3, wherein step (C-II) comprises: (C-II-3) contacting the precursor catalyst composition with an aqueous solution of NH₄F·HF; and subsequently (C-II-4) washing and drying the contacted precursor catalyst composition.

C7. The process of any of C1 to C6, wherein step (C-III) comprises: (C-III-1) combining the treated precursor catalyst composition with an auxiliary component; and (C-III-2) obtaining the isomerization catalyst composition from the combined mixture from step (C-III-1).

C8. The process of C7, wherein the auxiliary component comprises a binder.

C9. The process of any of C1 to C8, wherein the precursor catalyst composition comprises a zeolite.

C10. The process of C9, wherein the zeolite comprises one or more of ZSM-5, ZSM-11, ZSM-5 and ZSM-11 intergrowth, ZSM-22, ZSM-23, ZSM-48, a MWW framework zeolite such as MCM-22, 36, 49, 56, and mixtures and combinations thereof.

C11. The process of C9 or C10, wherein the zeolite has a silica (SiO₂) to alumina (Al₂O₃) molar ratio of 10 to 100, a total surface area of 200 m²/g to 700 m²/g, and a micropore surface area of 50 m²/g to 600 m²/g.

C12. The process of any of C1 to C11, wherein the precursor catalyst composition exhibits an external surface area of less than 55 m²/g.

C13. The process of any of C1 to C12, wherein the isomerization catalyst composition comprises a zeolite having one or more of the following features: a silica (SiO₂) to alumina (Al₂O₃) molar ratio of r1 to r2, where r1 and r2 can be, independently, e.g., 10, 15, 20, 25, 30, 35, 40, 45, 50, 55, 60, 65, 70, 75, 80, 85, 90, 95, 100, as long as r1<r2; a total surface area of s(t)1 to s(t)2 m²/g, where s(t)1 and s(t)2 can be, independently, e.g., 200, 250, 300, 350, 400, 450, 500, 550, 600, 650, 700, as long as s(t)1<s(t)2; a micropore surface area of s(mp)1 to s(mp)2 m²/g, where s(mp)1 and s(mp)2 can be, independently, e.g., 50, 100, 150, 200, 250, 300, 350, 400, 450, 500, 550, 600; and an external surface area of s(e)1 to s(e)2 m²/g, where s(e)1 and s(e)2 can be, independently, e.g., 55, 60, 65, 70, 75, 80, 85, 90, 95, 100, 120, 140, 150, 160, 180, 200, 220, 240, 250, 260, 280, 300, 350, 400, 450, 500, 550, as long as se1<se2.

C14. The process of any of C1 to C13, wherein the precursor catalyst composition comprises a binder.

C15. The process of C14, wherein the isomerization catalyst composition comprises the binder.

C16. The process of C14 or C15, wherein the binder is selected from silica, alumina, zirconia, a titanium oxide, a thorium oxide, yttria, a chromium oxide, a manganese oxide, hafnia, a lanthanide oxide, an alkali metal oxide, an alkaline earth metal oxide, and combinations, mixtures, and compounds thereof.

C17. The process of any of C14 to C16, wherein the isomerization catalyst composition comprises the binder at an amount of from c(b)1 to c(b)2 wt %, based on the total weight of the isomerization catalyst composition, where c(b)1 and c(b)2 can be, independently, e.g., 1, 2, 3, 4, 5, 6, 7, 8, 9, 10, 15, 20, 25, 30, 35, 40, 45, 50, 55, 60, 65, 70, 75, 80, 85, 90, 95, as long as c(b)1<c(b)2.

C18. The process of any of C1 to C17, wherein the conversion conditions comprises at least one of the following: (i) a temperature in a range from T1 to T2° C., where T1 and T2 can be, independently, e.g., 160, 170, 180, 190, 200, 210, 220, 230, 240, 250, 260, 270, 280, 300, 350, as long as T1<T2; (ii) an absolute pressure in a range from p1 to p2 kilopascal, where p1 and p2 can be, independently, e.g., 100, 200, 300, 400, 500, 600, 700, 800, 900, 1000, 1500, 2000, 3000, 4000, 5000, as long as p1<p2; (iii) a H2 concentration in the hydrocarbon feed in a range from c(H2)1 to c(H2)2 ppm by weight, based on the total weight of the hydrocarbon feed, where c(H2)1 and c(H2)2 can be, independently, e.g., 0, 1, 3, 4, 5, 6, 7, 8, 9, 10, 15, 20, 25, 30, 40, 50, 60, 70, 80, 90, 100, 150, 200, 250, 300, 350, 400, 450, 500, as long as c(H2)1<c(H2)2; and (iv) a WHSV of the hydrocarbon feed in a range from w1 to w2 hr⁻¹, where w1 and w2 can be, independently, e.g., 0.5, 1, 1.5, 2, 2.5, 3, 3.5, 4, 5, 6, 7, 8, 9, 10, 11, 12, 13, 14, 15, 16, 17, 18, 19, 20, as long as w1<w2.

Certain embodiments and features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

1. A process for converting a hydrocarbon teed comprising C8 aromatic hydrocarbons, the process comprising:
(I) feeding the hydrocarbon feed into a conversion zone; and
(II) contacting the hydrocarbon feed at least partly in a liquid phase with an isomerization catalyst composition in the conversion zone under conversion conditions to effect isomerization of at least a portion of the C8 aromatic hydrocarbons to produce a conversion product rich in para-xylene, wherein the isomerization catalyst composition comprises a zeolite having a silica (SiO₂) to alumina (Al₂O₃) molar ratio of 10 to 100, a total surface area of 200 m²/g to 700 m²/g, a micropore surface area of 50 m²/g to 600 m²/g, and an external surface area of 55 m²/g to 550 m²/g.

2. The process of claim 1, wherein the silica (SiO₂) to alumina (Al₂O₃) molar ratio is 15 to 60.

3. The process of claim 1, wherein the total surface area is 300 m²/g to 600 m²/g, the micropore surface area is 200 m²/g to 550 m²/g, and the external surface area is 60 m²/g to 350 m²/g.

4. The process of claim 1, wherein the zeolite is a ZSM-5 zeolite.

5. The process of claim 1, wherein the isomerization catalyst composition is an extrudate comprising the ZSM-5 zeolite and a binder.

6. The process of claim 1, wherein the silica (SiO₂) to alumina (Al₂O₃) molar ratio is 15 to 60, and the external surface area is 80 m²/g to 350 m²/g.

7. The process of claim 1, wherein the silica (SiO₂) to alumina (Al₂O₃) molar ratio is 20 to 40, and the external surface area is 100 m²/g to 200 m²/g.

8. The process of claim 1, wherein the LSM-5 zeolite is in the form of a LSM-5/LSM-11 intergrowth zeolite.

9. The process of claim 1, wherein the isomerization catalyst composition comprises from 1 wt % to 100 wt % of the ZSM-5 zeolite, based on a total weight of all zeolites present in the isomerization catalyst composition.

10. The process of claim 1, wherein:
the isomerization catalyst composition is an extrudate comprising the ZSM-5 zeolite and a binder,
the binder comprises silica, alumina, or a mixture thereof, and
the extrudate comprises 10 wt % to 90 wt % of the binder, based on the combined weight of the ZSM-5 zeolite and the binder.

11. The process of claim 1, wherein the conversion conditions comprise an absolute pressure sufficient to maintain the C8 aromatic hydrocarbons in liquid phase, and wherein the conversion conditions comprise a weight hour space velocity of 0.1 hr⁻¹ to 20 hr⁻¹ and a temperature of 140° C. to 400° C.

12. The process of claim 1, wherein the conversion conditions comprise an absolute pressure sufficient to maintain the C8 aromatic hydrocarbons in liquid phase, and wherein the conversion conditions comprise a weight hour space velocity of 4 hr⁻¹ to 12 hr⁻¹ and a temperature of 200° C. to 280° C.

13. The process of claim 1, wherein molecular hydrogen is fed into the conversion zone, and wherein the molecular hydrogen is present in an amount of 4 ppm to 250 ppm, based on the weight of the hydrocarbon feed.

14. The process of claim 1, wherein molecular hydrogen is not fed into the conversion zone.

15. The process of claim 1, wherein the conversion conditions comprise an absolute pressure sufficient to maintain the C8 aromatic hydrocarbons in liquid phase, and wherein, when the hydrocarbon teed comprises less than 5 wt % of para-xylene, the process exhibits a para-xylene selectivity of at least 16% at a weight hour space velocity of 2.5 hr⁻¹, 5 hr⁻¹ and 10 hr⁻¹.

16. A process for converting an aromatic hydrocarbon, comprising:
(I) feeding a hydrocarbon feed comprising C8 aromatic hydrocarbons into a conversion zone; and
(II) contacting the hydrocarbon feed with a catalyst comprising a ZSM-5 zeolite in the conversion zone under conversion conditions to effect isomerization of at least a portion of the C8 aromatic hydrocarbons to produce a conversion product rich in para-xylene, wherein:
the conversion conditions comprise an absolute pressure sufficient to maintain the C8 aromatic hydrocarbons at least partly in a liquid phase, a weight hour space velocity of 1 hr⁻¹ to 15 hr⁻¹, and a temperature of 150° C. to 300° C., and
the isomerization catalyst composition comprises a ZSM-5 zeolite having a silica (SiO₂) to alumina (Al₂O₃) molar ratio of 20 to 40, a total surface area of 400 m²/g to 500 m²/g, a micropore surface area of 300 m²/g to 450 m²/g, and an external surface area of 100 m²/g to 200 m²/g.

17. The process of claim 16, wherein, when the hydrocarbon feed comprises less than 5 wt % of para-xylene, the process exhibits a para-xylene selectivity of at least 19% at a weight hour space velocity of 2.5 hr⁻¹, 5 hr⁻¹, and 10 hr⁻¹.

18. A process for converting a hydrocarbon feed comprising C8 aromatic hydrocarbons, the process comprising:
(I) providing a precursor catalyst composition exhibiting a first external surface area of a1 m²/g;
(II) treating the precursor catalyst composition to obtain a treated precursor catalyst composition, wherein the treated precursor catalyst composition exhibits a second external surface area of a2 m²/g, and wherein (a2−a1)/a1*100%≥10%;
(III) forming an isomerization catalyst composition from the treated precursor catalyst composition;
(IV) feeding the hydrocarbon feed into a conversion zone; and
(V) contacting the hydrocarbon feed at least partly in a liquid phase with the isomerization catalyst composition in the conversion zone under conversion conditions to effect isomerization of at least a portion of the C8 aromatic hydrocarbons to produce a conversion product rich in para-xylene.

19. The process of claim 18, wherein 20%≤(a2−a1)/a1*100%≤1000%.

20. The process of claim 18, wherein step (V) exhibits a para-xylene selectivity of sel(pX)2 wt %, and a reference step (V-ref) below exhibits a para-xylene selectivity of sel(pX)1 wt %:
(V-ref) contacting the hydrocarbon feed at least partly in a liquid phase with the precursor catalyst composition in the conversion zone under the same conversion conditions in step (V) to effect isomerization of at least a portion of the C8 aromatic hydrocarbons to produce a reference conversion product rich in para-xylene; wherein
$$y 1 \% \leq \frac{( sel ( p X ) 2 - sel ( p X ) 1}{sel ( p X ) 1} \times 100 \% \leq y 2 \% ,$$
wherein y1=5 and y2=1000.

21. The process of claim 18, wherein step (II) comprise:
(II-1) contacting the precursor catalyst composition with an alkaline aqueous solution; and subsequently
(II-2) washing and drying the contacted precursor catalyst composition.

22. The process of claim 21, wherein the alkaline aqueous solution comprises LiOH, NaOH, KOH, RbOH, CsOH, Na₂CO₃, Mg(OH)₂, Ca(OH)₂, Sr(OH)₂, and mixtures thereof

23. The process of claim 18, wherein step (II) comprise:
(II-3) contacting the precursor catalyst composition with an aqueous solution of NH₄F·HF; and subsequently
(II-4) washing and drying the contacted precursor catalyst composition.

24. The process of claim 18, wherein step (III) comprises:
(III-1) combining the treated precursor catalyst composition -with an auxiliary component, and
(III-2) obtaining the isomerization catalyst composition from the combined mixture from step (C-III-1).

25. The process of claim 24, wherein at least one of the following is met:
(i) the precursor catalyst composition comprises ZSM-5; and
(ii) the auxiliary component comprises a binder.
